(19) United States
(12) Patent Application Publication
(10) Pub. No.: US 2026/0218182 A1
(43) Pub. Date: Jul. 30, 2026

(54) THERAPEUTIC OLIGONUCLEOTIDE-CONTAINING PHARMACEUTICAL COMPOSITIONS AND DOSING REGIMENS USING THE SAME

(71) Applicant: ELI LILLY AND COMPANY, Indianapolis, IN (US)

(72) Inventors: Ashish GARG, Carmel, IN (US); Amritpreet KAUR, Whitestown, IN (US); John Henry KREGE, Durham, NC (US); Xiaosu MA, Mercer Island, WA (US); Wei NI, Indianapolis, IN (US); Helle Linnebjerg HAW, Grand Rapids, MI (US)

(21) Appl. No.: 19/143,747

(22) PCT Filed: Jan. 12, 2024
(86) PCT No.: PCT/US2024/011378

§ 371 Date: Jun. 26, 2025

Related U.S. Application Data
(60) Provisional application No. 63/479,757, filed on Jan. 13, 2023.

(51) Int. Cl. C12N 15/113; A61P 9/10

(52) U.S. Cl. CPC C12N 15/113; A61P 9/10; C12N 2310/14; C12N 2320/35

(57) ABSTRACT

Pharmaceutical compositions are disclosed that include a double-stranded RNAi agent that modulates LPA expression or its pharmaceutically acceptable salt thereof in water. Doses and dosing regimens for such RNAi agents also are disclosed that include administering doses from about 4 mg to about 608 mg at a frequency of monthly to yearly.

### REFERENCE TO SEQUENCE LISTING SUBMITTED ELECTRONICALLY

The disclosure is being filed along with a Sequence Listing in ST.26 XML format. The Sequence Listing is provided as a file titled “30459_US_PRI” created 5 Dec. 2022 and is 28 kilobytes (kb) in size. The Sequence Listing information in the ST.26 XML format is incorporated herein by reference in its entirety.

### TECHNICAL FIELD

This disclosure generally relates to biology and medicine, and more specifically it relates to pharmaceutical compositions such as formulations having therapeutic oligonucleotides (e.g., ds RNAi agents) that modulate apolipoprotein(a) gene (LPA) expression and relates to dosing regimens using the same for attenuating, preventing and/or treating diseases, disorders and/or conditions associated with LPA expression.

### BACKGROUND

Lipoprotein(a) (Lp(a)) is a heterogeneous low-density lipoprotein (LDL)-like particle containing a lipid core with apolipoprotein B-100 (ApoB) and apolipoprotein(a) (Apo(a)), where Apo(a) is attached to ApoB through a disulfide bond. LPA is expressed predominantly in the liver and is restricted to human and Old World non-human primates. Lp(a) levels in humans are genetically defined and do not change significantly with diet, exercise or other lifestyle changes.

Normal Lp(a) levels range from 0.1 mg/dL to 25 mg/dL, with about 25% of the population in the United States having Lp(a) levels of 30 mg/dL or higher. Analysis of Lp(a) levels in multiple studies have implicated high Lp(a) levels as an independent risk factor for cardiovascular disease, stroke and other related disorders. When therapeutic lipoprotein apheresis is used to lower both Lp(a) and LDL levels in hyperlipidemic individuals, significant reductions of cardiovascular events have been observed.

In view thereof, there is a need for safe and effective formulations including therapeutic oligonucleotides that modulate LPA expression and for dosing regimens using the same for attenuating, preventing and/or treating diseases, disorders and/or conditions associated with LPA expression, while also preserving an overall acceptable benefit/risk profile to the individual.

### BRIEF SUMMARY

To address this need, the disclosure describes doses of a therapeutic oligonucleotide that modulates LPA expression, where a dose thereof can be from about 4 mg to about 608 mg. In some instances, the therapeutic oligonucleotide is a RNAi agent, especially a double-stranded (ds) RNAi agent. In certain instances, the ds RNAi agent has a sense strand and an antisense strand, where the sense strand includes a nucleotide sequence of SEQ ID NO:1 or 3 and where the antisense strand includes a nucleotide sequence of SEQ ID NO:2 or 4. Such doses can be used in attenuating, preventing and/or treating diseases, disorders and/or conditions associated with LPA expression.

In some instances, the dose can be from about 4 mg to about 608 mg. In other instances, the dose can be about 4 mg, about 12 mg, about 16 mg, about 32 mg, about 64 mg, about 96 mg, about 128 mg, about 160 mg, about 192 mg, about 224 mg, about 256 mg, about 288 mg, about 304 mg, about 320 mg, about 352 mg, about 384 mg, about 400 mg, about 416 mg, about 448 mg, about 480 mg, about 512 mg, about 544 mg, about 576 mg or about 608 mg.

In addition, and to facilitate administering the doses herein to an individual in need thereof, the disclosure describes pharmaceutical compositions such as formulations that include at least the ds RNAi agent herein, or a pharmaceutically acceptable salt thereof, that modulates LPA expression. Such formulations can be used in attenuating, preventing and/or treating diseases, disorders and/or conditions associated with LPA expression. For example, the formulations can be used to reduce the risk of major adverse cardiovascular events (MACE) in individuals with or at high risk of atherosclerotic cardiovascular disease (ASCVD) and/or high Lp(a) levels (e.g., >125 nmol/L; 50 mg/dL) or in individuals at risk for a first cardiovascular (CV) event. Moreover, such formulations can be packaged for intravenous (IV) or subcutaneous (SC) administration as described herein with maintenance of, for example, product stability and other desirable attributes.

In particular, pharmaceutical compositions are described that include the ds RNAi agent herein or a pharmaceutically acceptable salt thereof in water (H₂O).

In some instances, the ds RNAi agent can be at a concentration from about 100 mg/mL to about 300 mg/mL. In other instances, the ds RNAi agent can be at a concentration from about 150 mg/mL to about 170 mg/mL, especially about 160 mg/mL, or from about 190 mg/mL to about 210 mg/mL, especially about 200 mg/mL.

In some instances, the formulations can be at a pH from about 6.0 to about 8.0. In other instances, the formulations can be at a pH of about 7.0.

In some instances, the pharmaceutical compositions herein can be preservative free.

In some instances, the pharmaceutical compositions herein can be diluted with about 0.9% NaCl injection for lower doses.

In one particular instance, a pharmaceutical composition includes a ds RNAi agent that modulates LPA expression, where the ds RNAi agent has a sense strand having a nucleotide sequence of SEQ ID NO:1 and an antisense strand having a nucleotide sequence of SEQ ID NO:2 in H₂O at a concentration from about 150 mg/mL to about 170 mg/mL, especially about 160 mg/mL, at pH 7.0, and where the pharmaceutical composition is preservative free.

In another instance, a pharmaceutical composition includes a ds RNAi agent that modulates LPA expression, where the ds RNAi agent has a sense strand having a nucleotide sequence of SEQ ID NO:1 and an antisense strand having a nucleotide sequence of SEQ ID NO:2 in H₂O at a concentration from about 190 mg/mL to about 210 mg/mL, especially about 200 mg/mL, at pH 7.0, and where the pharmaceutical composition is preservative free.

In one particular instance, a pharmaceutical composition includes a ds RNAi agent that modulates LPA expression, where the ds RNAi agent has a sense strand having a nucleotide sequence of SEQ ID NO:3 and an antisense strand having a nucleotide sequence of SEQ ID NO:4 in H₂O at a concentration from about 150 mg/mL to about 170 mg/mL, especially about 160 mg/mL, at pH 7.0, and where the pharmaceutical composition is preservative free.

In another particular instance, a pharmaceutical composition includes a ds RNAi agent that modulates LPA expression, where the ds RNAi agent has a sense strand having a nucleotide sequence of SEQ ID NO:3 and an antisense strand having a nucleotide sequence of SEQ ID NO:4 in H₂O at a concentration from about 190 mg/mL to about 210 mg/mL, especially about 200 mg/mL, at pH 7.0, and where the pharmaceutical composition is preservative free.

The disclosure also describes dosing regimens for and methods of attenuating, preventing and/or treating diseases, disorders and/or conditions associated with LPA expression, where such methods include a step of administering to an individual in need thereof a first dose of a ds RNAi agent that modulates LPA expression or a pharmaceutical composition thereof such as a formulation herein, where the dose can be from about 4 mg to about 608 mg.

In some instances, the ds RNAi agent includes a sense strand having a nucleotide sequence of SEQ ID NO:1 and an antisense strand having a nucleotide sequence of SEQ ID NO:2. In other instances, the ds RNAi agent includes a sense strand having a nucleotide sequence of SEQ ID NO:3 and an antisense strand having a nucleotide sequence of SEQ ID NO:4.

In some instances, the first dose can be about 4 mg, about 12 mg, about 16 mg, about 32 mg, about 64 mg, about 96 mg, about 128 mg, about 160 mg, about 192 mg, about 224 mg, about 256 mg, about 288 mg, about 304 mg, about 320 mg, about 352 mg, about 384 mg, about 400 mg, about 416 mg, about 448 mg, about 480 mg, about 512 mg, about 544 mg, about 576 mg or about 608 mg.

In some instances, the methods include a step of administering one or more subsequent doses (i.e., second, third, fourth, fifth, et seq. dose) about every month (Q1M), about every three months (Q3M), about every six months (Q6M), about every nine months (Q9M) or about every twelve months (Q12M) from the first dose (or from a previous dose for any other subsequent dose), where the one or more subsequent doses can be from about 4 mg to about 608 mg.

In some instances, the one or more subsequent doses can be about 4 mg, about 12 mg, about 16 mg, about 32 mg, about 64 mg, about 96 mg, about 128 mg, about 160 mg, about 192 mg, about 224 mg, about 256 mg, about 288 mg, about 304 mg, about 320 mg, about 352 mg, about 384 mg, about 400 mg, about 416 mg, about 448 mg, about 480 mg, about 512 mg, about 544 mg, about 576 mg or about 608 mg.

In some instances, the one or more subsequent doses can be the same as the first dose. In other instances, the one or more subsequent doses can be different from the first dose. In yet other instances, a second dose can be the same as the other subsequent doses (but all different from the first dose). In yet other instances, the second dose can be different from the other subsequent doses (but not all are necessarily different from the first dose).

In one particular instance, the second dose can be administered about 1 month, about 3 months, about 6 months, about 9 months or about 12 months after the first dose, and any other subsequent doses (i.e., third, fourth, fifth, et seq. dose) can be administered about Q6M, about Q9M or about Q12M from the second dose or from a previous dose thereafter. In another particular instance, the second dose can be administered about 6 months after the first dose, and any other subsequent doses (i.e., third, fourth, fifth, et seq. dose) can be administered about Q6M from the second dose or from a previous dose thereafter. In another particular instance, the second dose can be administered about 6 months after the first dose, and any other subsequent doses (i.e., third, fourth, fifth, et seq. dose) can be administered about Q12M from the second dose or from a previous dose thereafter. In another particular instance, the second dose can be administered about 12 months after the first dose, and any other subsequent doses (i.e., third, fourth, fifth, et seq. dose) can be administered about Q12M from the second dose or from a previous dose thereafter. In another particular instance, the second dose can be administered about 6 months after the first dose, the third dose can be administered about 6 months after the second dose, and any other subsequent doses (i.e., fourth, fifth, et seq. dose) can be administered about Q12M from the third dose or from a previous dose thereafter.

Alternatively, the disclosure describes methods of reducing LPA expression, where such methods include a step of administering to an individual in need thereof a first dose of a ds RNAi agent that modulates LPA expression or a pharmaceutical composition thereof such as a formulation herein, where the dose can be from about 4 mg to about 608 mg.

In some instances, the ds RNAi agent includes a sense strand having a nucleotide sequence of SEQ ID NO:1 and an antisense strand having a nucleotide sequence of SEQ ID NO:2. In other instances, the ds RNAi agent includes a sense strand having a nucleotide sequence of SEQ ID NO:3 and an antisense strand having a nucleotide sequence of SEQ ID NO:4.

In some instances, the first dose can be about 4 mg, about 12 mg, about 16 mg, about 32 mg, about 64 mg, about 96 mg, about 128 mg, about 160 mg, about 192 mg, about 224 mg, about 256 mg, about 288 mg, about 304 mg, about 320 mg, about 352 mg, about 384 mg, about 400 mg, about 416 mg, about 448 mg, about 480 mg, about 512 mg, about 544 mg, about 576 mg or about 608 mg.

In some instances, the methods include a step of administering one or more subsequent doses (i.e., second, third, fourth, fifth, et seq. dose) about every Q1M, about every Q3M, about every Q6M, about every Q9M or about every Q12M following the first dose (or from a previous dose in the case of a third, fourth, fifth, et seq. dose), where the one or more subsequent doses can be from about 4 mg to about 608 mg.

In some instances, the one or more subsequent doses can be about 4 mg, about 12 mg, about 16 mg, about 32 mg, about 64 mg, about 96 mg, about 128 mg, about 160 mg, about 192 mg, about 224 mg, about 256 mg, about 288 mg, about 304 mg, about 320 mg, about 352 mg, about 384 mg, about 400 mg, about 416 mg, about 448 mg, about 480 mg, about 512 mg, about 544 mg, about 576 mg or about 608 mg.

In some instances, the one or more subsequent doses can be the same as the first dose. In other instances, the one or more subsequent doses can be different from the first dose. In yet other instances, a second dose can be the same as the other subsequent doses (but all different from the first dose). In yet other instances, the second dose can be different from the other subsequent doses (but not all are necessarily different from the first dose).

In one particular instance, the second dose can be administered about 1 month, about 3 months, about 6 months or about 12 months after the first dose, and any other subsequent doses (i.e., third, fourth, fifth, et seq. dose) can be administered about Q6M, Q9M or about Q12M from the second dose or from a previous dose thereafter. In another particular instance, the second dose can be administered about 6 months after the first dose, and any other subsequent doses (i.e., third, fourth, fifth, et seq. dose) can be administered about Q6M from the second dose or from a previous dose thereafter. In another particular instance, the second dose can be administered about 6 months after the first dose, and any other subsequent doses (i.e., third, fourth, fifth, et seq. dose) can be administered about Q12M from the second dose or from a previous dose thereafter. In another particular instance, the second dose can be administered about 12 months after the first dose, and any other subsequent doses (i.e., third, fourth, fifth, et seq. dose) can be administered about Q12M from the second dose or from a previous dose thereafter. In another particular instance, the second dose can be administered about 6 months after the first dose, the third dose can be administered about 6 months after the second dose, and any other subsequent doses (i.e., fourth, fifth, et seq. dose) can be administered about Q12M from the third dose or from a previous dose thereafter.

In any of the above methods, the administering can be by IV administration or by SC administration.

In any of the above methods, the individual can have a confirmed disease, disorder and/or condition associated with LPA expression. In some instances, the individual can be at risk for a disease, disorder and/or condition associated with LPA expression, especially risk of major CV events, including myocardial infarction (MI), stroke, coronary revascularization, peripheral vascular events and CV mortality. In other instances, the individual can be an adult with elevated Lp(a) and established CV disease. In yet other instances, the individual can be an adult at risk for a first CV event and optionally with elevated Lp(a).

In any of the above methods, the diseases, disorders and/or conditions associated with LPA expression include atherosclerosis, calcific aortic valve stenosis (CAVS), cardiometabolic diseases, cardiovascular events including coronary revascularization, CV mortality, cerebrovascular disease, chronic kidney disease, coronary heart disease, dyslipidemia, heart failure, MI and stroke, and/or peripheral vascular disease.

In any of the above methods, the individual can have a Lp(a) level of at least about >75 nmol/L. In some instances, the individual can have a Lp(a) level of at least about 100 nmol/L, about 125 nmol/L, about 150 nm/L, about 175 nmol/L or about 200 nmol/L prior to administering the first dose.

In any of the above methods, the individual can have a single-nucleotide polymorphism (SNP) of LPA selected from rs10455872 and rs3798220.

The disclosure further describes a composition herein for use as a medicament.

The disclosure further describes a composition herein for use in the treatment of diseases, disorders and/or conditions associated with LPA expression.

The disclosure further describes an article of manufacture including a formulation herein. In some instances, the article of manufacture is a single-use vial or a multi-use vial. In some instances, the article of manufacture is a pre-filled syringe. In some instances, the article of manufacture is an automatic injection apparatus. In some instances, the article of manufacture is a pump for continuous perfusion, especially a pump for subcutaneous infusion.

An advantage of the doses, regimens, methods and uses herein is that they provide a limited duration of exposure, durable treatment effects and fewer adverse events (AEs). Likewise, such doses, regimens, methods and uses have a potential to improve acceptance and compliance in view of their infrequent administration that is required.

### DETAILED DESCRIPTION

### Overview

Lp(a) is considered a strong genetic risk factor for coronary heart disease. Genetic variation within LPA, which determines the level of Apo(a) production, is strongly associated with both Lp(a) levels in plasma and risk of MI.

Mechanistically, increased plasma Lp(a) levels may act to increase the risk of cardiovascular disease (CVD) by its atherogenic, inflammatory and possibly prothrombotic effects. Accordingly, three non-mutually exclusive mechanisms have been proposed for Lp(a)'s role in CVD: (1) Lp(a) may accelerate atherogenesis as a result of intimal deposition of Lp(a) cholesterol oxidized phospholipids, or both, (2) Lp(a) contains oxidized phospholipids that are pro-inflammatory, and (3) Apo(a) possesses structural similarity with plasminogen (PLG) and plasmin but has no fibrinolytic activity, thereby potentially reducing fibrinolysis by competing for fibrin binding.

Intl. Patent Application Publication No. WO 2022/032288 describes a ds RNAi agent (e.g., LPA-3291-Ml) that can be used for attenuating, preventing and/or treating diseases, disorders and/or conditions associated with LPA expression (i.e., reduce the levels of LPA mRNA and Apo(a) to thereby decrease Lp(a) activity/level).

It is surprisingly shown herein that doses of the ds RNAi agent and formulations including the same rapidly decrease Lp(a) (e.g., within about 4 days to about 8 days depending upon dose) after a single dose, decrease Lp(a) by >90% within about 14 days after a single dose, and are durable out to >24 weeks after a single dose. At higher doses, the ds RNAi agent and formulations including the same decrease Lp(a) by 70%-90% out to 48 weeks. Moreover, the doses herein showed no dose-dependent changes in PLG activity.

### Abbreviations and Definitions

Unless defined otherwise, all technical and scientific terms used herein have the same meaning as commonly understood by one of skill in the art to which the disclosure pertains. Although any methods and materials similar to or equivalent to those described herein can be used in the practice or testing of the methods herein, the preferred methods and materials are described herein.

Additionally, reference to an element by the indefinite article “a” or “an” does not exclude the possibility that more than one element is present, unless the context clearly requires that there be one and only one element. The indefinite article “a” or “an” thus usually means “at least one.”

Moreover, use of “including,” as well as other forms, such as “including but not limited, “include,” “includes” and “included,” is not limiting.

Certain abbreviations used herein are as follows:

“A2AP” refers to alpha 2-antiplasmin; “AE” refers to adverse event; “Apo(a)” refers to apolipoprotein(a); “ApoB” refers to apolipoprotein B-100; “ASCVD” refers to atherosclerotic cardiovascular disease; “AUC” refers to area under the plasma concentration versus time curve; “bp” refers to base pair(s); “CAC” refers to coronary artery calcium; “CAD” refers to coronary artery disease; “CAVS” refers to calcific aortic valve stenosis; “CK” refers to creatine kinase; “Cₘₐₓ” refers to maximum plasma concentration; “CVD” refers to cardiovascular disease; “ECG” refers to electrocardiogram; “HCl” refers to hydrochloric acid; “H₂O” refers to water; “hsCRP” refers to high-sensitivity C-reactive protein; “IV” refers to intravenous or intravenously; “LDL” refers to low-density lipoprotein; “LDL-C” refers to low-density lipoprotein cholesterol; “LPA” refers to apolipoprotein(a) gene; “Lp(a)” refers to lipoprotein(a) protein; “MACE” refers to major adverse cardiovascular event(s); “MI” refers to myocardial infarction; “mRNA” refers to messenger ribonucleic acid; “NaOH” refers to sodium hydroxide; “NOAEL” refers to no-observed-adverse-effect level; “NOEL” refers to no-observed-effect level; “nt” refers to nucleotide(s); “PAD” refers to peripheral artery disease; “Pal-i” refers to plasminogen activator inhibitor-1; “PD” refers to pharmacodynamics; “PK” refers to pharmacokinetics; “PLG” refers to plasminogen; “PO” refers to phosphodiester; “PS” refers to phosphorothioate; “RISC” refers to RNA-induced silencing complex; “SAE” refers to serious adverse event; “SC” refers to subcutaneous or subcutaneously; “SNP” refers to single-nucleotide polymorphism(s); “t i” refers to half-life; “TC” refers to total cholesterol; “TEAE” refers to treatment-emergent adverse event; “TG” refers to triglyceride(s); “tPA” refers to tissue plasminogen activator; “tₘₐₓ” refers to time of maximum observed concentration; and “WFI” refers to water-for-injection.

Certain definitions used herein are defined as follows:

As used herein, “about” means within a statistically meaningful range of a value or values such as, for example, a stated activity, concentration, dose, length, molecular weight, pH, sequence similarity, time frame, temperature, volume, etc. Such a value or range can be within an order of magnitude typically within 20%, more typically within 10%, and even more typically within 5% of a given value or range. The allowable variation encompassed by “about” will depend upon the particular system under study, and can be readily appreciated by one of skill in the art.

As used herein, “administer,” “administering,” “administration” and the like mean providing a substance (e.g., a ds RNAi agent herein or a pharmaceutical composition herein) to an individual in a manner that is pharmacologically useful (e.g., to attenuate, prevent and/or treat a disease, disorder and/or condition in the individual).

As used herein, “at risk for a first cardiovascular event” or “at risk for a first CV event” means an individual having (1) documented coronary artery disease (CAD), carotid stenosis or peripheral artery disease (PAD) without history of event or revascularization, or (2) known familial hypercholesteremia. Alternatively, “at risk for a first cardiovascular event” or “at risk for a first CV event” means an individual having at least three high-risk factors selected from elevated coronary artery calcium (CAC; e.g., >100 for individuals assigned female at birth and <65 years of age, or >300 for anyone) score, current tobacco use, diabetes, older age (e.g., ≥70 years old for individuals assigned female at birth or ≥65 years old for individuals assigned male at birth), kidney disease, hypertension, high-sensitivity C-reactive protein (hsCRP; e.g., ≥2 mg/L), family history of premature ASCVD and hyperlipidemia (e.g., low-density lipoprotein cholesterol (LDL-C)≥100 mg/dL). The individual additionally may have an elevated Lp(a) level (>75 nmol/L, such as, for example, ≥175 nmol/L).

As used herein, “attenuate,” “attenuating,” “attenuation” and the like mean that a qualitative or quantitative measure of signs and/or symptoms and/or biomarkers of a given disease, disorder and/or condition can be decreased in an individual by about 5%, 10%, 15%, 20%, 25%, 30%, 35%, 40%, 45%, 50%, 55%, 60%, 65%, 70%, 75%, 80%, 85%, 90%, 95% or 100% when administered a substance (when compared to an appropriate control). Although there can be overlap between “attenuating” and “preventing,” it is intended that the latter is a more drastic (i.e., less subtle) decrease in the signs and/or symptoms and/or biomarkers. For example, a disease, disorder and/or condition is “attenuated” if existing signs and/or symptoms and/or biomarkers are reduced in intensity and/or frequency but may not completely disappear.

As used herein, “chemical stability” means an ability of a substance (e.g., a ds RNAi agent herein or a pharmaceutical composition herein) to resist potential changes in composition in the product due to chemical reactions that may occur, such as aggregation, fragmentation, hydrolysis, isomerization, oxidation and polymerization.

As used herein, “disease, disorder and/or condition associated with LPA expression” and the like mean atherosclerosis, CAVS, cardiometabolic diseases, cardiovascular events including coronary revascularization, CV mortality, cerebrovascular disease, chronic kidney disease, coronary heart disease, dyslipidemia, heart failure, MI and stroke, and/or peripheral vascular disease.

As used herein, “dose” or “doses” means a quantity of a substance (e.g., a ds RNAi agent herein or a pharmaceutical composition herein) that is administered to an individual in a discrete amount at a particular point in time. When used in connection with the terms dose, doses, dosing and the like, “adjustment” or “adjusting” mean a quantity of any decrease or increase to the prior-administered dose. When used in connection with the terms dose, doses, dosing and the like, “regimen” means a set of guidelines for determining and administering one or more doses and/or adjustments thereto.

As used herein, “effective amount” means an amount, concentration or dose of one or more substances (e.g., a ds RNAi agent herein or a pharmaceutical composition herein) that, upon single or multiple dose administration to an individual in need thereof, provides a desired effect in such an individual under diagnosis or treatment (i.e., may produce a clinically measurable difference in a status of the individual). An effective amount can be readily determined by one of skill in the art by using known techniques and by observing results obtained under analogous circumstances. In determining the effective amount for an individual, a number of factors are considered, including, but not limited to, the species of the individual, its size, age and general health, the specific disease, disorder and/or condition involved, the degree of or involvement or the severity of the disease, disorder and/or condition, the response of the individual, the particular active ingredient administered, the mode of administration, the bioavailability characteristics of the preparation administered, the dose regimen selected, the use of concomitant therapeutic agent(s), and other relevant circumstances.

As used herein, “individual” means any mammal, including cats, dogs, mice, rats, and primates (human and non-human), especially humans. Moreover, “participant,” patient” or “subject” may be used interchangeably with “individual.”

As used herein, “individual in need thereof” means a mammal, such as a human, with a disease, disorder and/or condition requiring treatment or therapy, including for example, those listed herein. In particular, the preferred individual to be treated is a human, especially an individual having or suspected of having a disease, disorder and/or condition associated with LPA expression.

As used herein, “medicament” means an active ingredient (e.g., a ds RNAi agent herein) to treat a disease, disorder and/or condition associated with LPA expression.

As used herein, “microbiological stability” means an ability of an active ingredient, substance or product to maintain its sterility when exposed to environmental or other microorganisms.

As used herein, “modulate,” “modulating,” “modulation” and the like means to change, affect or interfere with the functioning of the components of systems. For example, the ds RNAi agent herein modulates LPA expression by mediating degradation of LPA mRNA thereby causing reduced LPA mRNA, reduced Apo(a) (level/activity) and/or reduced Lp(a) (level/activity).

As used herein, “nucleotide” means an organic compound having a nucleoside (a nucleobase such as, for example, adenine, cytosine, guanine, thymine, or uracil; and a pentose sugar such as, for example, ribose or 2′-deoxyribose) and a phosphate group. A “nucleotide” can serve as a monomeric unit of nucleic acids such as deoxyribonucleic acid (DNA) oligonucleotides and ribonucleic acid (RNA) oligonucleotides.

As used herein, “oligonucleotide” means a short nucleic acid molecule (e.g., less than about 100 nucleotides in length), which may be single-stranded (ss) or ds.

As used herein, “pharmaceutical formulation” or “formulation” means a preparation that is in such form as to permit the biological activity of the active ingredient (e.g., a ds RNAi agent herein) to be effective and that contains no additional components having unacceptable toxicity to an individual to which the formulation would be administered. Such formulations are sterile. “Pharmaceutically acceptable” excipients (e.g., additives, vehicles, etc.) mean those that reasonably can be administered to an individual to provide an effective dose of the active ingredient employed.

As used herein, “preservative” means a compound that can be included in a formulation to essentially reduce bacterial action therein, thus facilitating the production of a multi-use formulation. Examples of preservatives include, but are not limited to, octadecyldimethylbenzyl ammonium chloride, hexamethonium chloride, benzalkonium chloride (a mixture of alkylbenzyldimethylammonium chlorides in which the alkyl groups are long-chain compounds) and benzethonium chloride. Other preservatives include aromatic alcohols such as phenol, butyl and benzyl alcohol, alkyl parabens such as methyl or propyl paraben, catechol, resorcinol, cyclohexanol, 3-pentanol and m-cresol.

As used herein, “preservative-free” or “preservative free” means a composition, such as a pharmaceutical composition (i.e., a formulation), that does not contain a preservative.

As used herein, “prevent,” “preventing,” “prevention” and the like mean that a qualitative or quantitative measure of signs and/or symptoms and/or biomarkers, of a given disease, disorder and/or condition can be decreased in an individual by about 5%, 10%, 15%, 20%, 25%, 30%, 35%, 40%, 45%, 50%, 55%, 60%, 65%, 70%, 75%, 80%, 85%, 90%, 95% or 100% when administered a substance (when compared to an appropriate control). Although there can be overlap between “attenuating” and “preventing,” it is intended that the latter is a more drastic (i.e., less subtle) decrease in the signs and/or symptoms and/or biomarkers. For example, a disease, disorder and/or condition is “prevented” if it does not appear. In this manner, some individuals can be considered (for diverse reasons, including genetics) as likely to develop a disease, disorder and/or condition, and can undergo a preventing regimen (i.e., prophylactic) according to avoid the disease, disorder and/or condition.

As used herein, “reduced expression,” and with respect to a gene (e.g., LPA) means a decrease in an amount or level of a RNA transcript (e.g., LPA mRNA) or protein (e.g., Apo(a)) encoded by the gene and/or a decrease in the amount or level of activity of the gene or its protein in a cell, a population of cells, a sample, an organ, a tissue, a system or an individual, when compared to an appropriate reference (e.g., a reference cell, population of cells, sample, organ, tissue, system or individual). For example, the act of contacting a cell with a substance (e.g., a ds RNAi agent herein or a pharmaceutical composition herein) may result in a decrease in the amount or level of mRNA, protein, and/or activity (e.g., via degradation of LPA mRNA by the RNAi pathway) when compared to a cell that is not treated with the substance. Similarly, and as used herein, “reducing expression” means an act that results in reduced expression of a gene (e.g., LPA). Specifically, and as used herein, “reduction of LPA expression” means a decrease in the amount or level of LPA mRNA, Apo(a) protein, and/or Lp(a) concentration and/or activity in a cell, a population of cells, a sample, or an individual when compared to an appropriate reference (e.g., a reference cell, population of cells, tissue, organ, system or individual).

As used herein, “iRNA,” “iRNA agent,” “RNAi,” “RNAi agent” and “RNA interference agent” means an oligonucleotide that contains RNA and that mediates the targeted cleavage of a RNA transcript via RNA interference, e.g., through a RNA-induced silencing complex (RISC) pathway. The RNAi agent can have a sense strand and an antisense strand, where the sense strand and the antisense strand form a duplex. In some instances, the sense and antisense strands of RNAi agent can be 21-23 nucleotides in length. Alternatively, the sense and antisense strands can be longer, for example, 25-36 nucleotides in length, in which case the longer nucleotide sequences are first processed by the Dicer enzyme. The RNAi agent directs sequence-specific degradation of mRNA via RNA interference. The RNAi agent attenuates, inhibits, modulates or reduces gene expression in a cell, tissue, organ, system or individual (e.g., LPA expression).

As used herein, “stable,” with regard to a formulation, means one in which the active ingredient therein (e.g., a ds RNAi agent herein) essentially retains its biological activity and/or chemical stability and/or physical stability upon storage. In this manner, the formulation essentially retains its chemical and physical stability, as well as retain its biological activity upon storage. The storage period generally can be based on an intended shelf-life of the formulation.

As used herein, “sterile,” with regard to a composition, such as a pharmaceutical composition or formulation, means aseptic or free or essentially free from all living microorganisms and spores.

As used herein, “strand” refers to a single, contiguous sequence of nucleotides linked together through internucleotide linkages (e.g., PO bonds/linkages or PS bond/linkages). A strand can have two free ends (e.g., a 5′ end and a 3′ end).

As used herein, “treat,” “treatment” or “treating” mean a process where there may be a slowing, controlling, delaying or stopping of the progression of the diseases, disorders or conditions disclosed herein, or ameliorating disease, disorder or condition symptoms, but does not necessarily indicate a total elimination of all disease, disorder or condition symptoms. Treatment and the like includes administration of a compound, composition or formulation as described herein for treatment of a disease, disorder and/or condition in an individual, particularly in a human.

### Doses and Pharmaceutical Compositions

The compositions herein include doses and pharmaceutical compositions such as formulations that include an effective amount of a ds RNAi agent that modulates LPA expression. Such doses and pharmaceutical compositions can be used in attenuating, preventing and/or treating diseases, disorders and/or conditions associated with LPA expression.

With regard to doses, a dose of the ds RNAi agent can be from about 4 mg to about 608 mg. In some instances, the dose can be from about 6 mg to about 606 mg, from about 8 mg to about 604 mg, from about 10 mg to about 602 mg, from about 12 mg to about 600 mg, from about 14 mg to about 598 mg, from about 16 mg to about 596 mg, from about 18 mg to about 594 mg, from about 20 mg to about 592 mg, from about 22 mg to about 590 mg, from about 24 mg to about 588 mg, from about 26 mg to about 586 mg, from about 28 mg to about 584 mg, from about 30 mg to about 582 mg, from about 32 mg to about 580 mg, from about 34 mg to about 578 mg, from about 36 mg to about 576 mg, from about 38 mg to about 574 mg, from about 40 mg to about 572 mg, from about 42 mg to about 570 mg, from about 44 mg to about 568 mg, from about 46 mg to about 566 mg, from about 48 mg to about 564 mg, from about 50 mg to about 562 mg, from about 52 mg to about 560 mg, from about 54 mg to about 558 mg, from about 56 mg to about 556 mg, from about 58 mg to about 554 mg, from about 60 mg to about 552 mg, from about 62 mg to about 550 mg, from about 64 mg to about 548 mg, from about 66 mg to about 546 mg, from about 68 mg to about 544 mg, from about 70 mg to about 542 mg, from about 72 mg to about 540 mg, from about 74 mg to about 538 mg, from about 76 mg to about 536 mg, from about 78 mg to about 534 mg, from about 80 mg to about 532 mg, from about 82 mg to about 530 mg, from about 84 mg to about 528 mg, from about 86 mg to about 526 mg, from about 88 mg to about 524 mg, from about 90 mg to about 522 mg, from about 92 mg to about 520 mg, from about 94 mg to about 518 mg, from about 96 mg to about 516 mg, from about 98 mg to about 514 mg, from about 100 mg to about 512 mg, from about 102 mg to about 510 mg, from about 104 mg to about 508 mg, from about 106 mg to about 506 mg, from about 108 mg to about 504 mg, from about 110 mg to about 502 mg, from about 112 mg to about 500 mg, from about 114 mg to about 498 mg, from about 116 mg to about 496 mg, from about 118 mg to about 494 mg, from about 120 mg to about 492 mg, from about 122 mg to about 490 mg, from about 124 mg to about 488 mg, from about 126 mg to about 486 mg, from about 128 mg to about 484 mg, from about 130 mg to about 482 mg, from about 132 mg to about 480, from about 134 mg to about 478 mg, from about 136 mg to about 476 mg, from about 138 mg to about 474 mg, from about 140 mg to about 472 mg, from about 142 mg to about 470 mg, from about 144 mg to about 468 mg, from about 146 mg to about 466 mg, from about 148 mg to about 464 mg, from about 150 to about 462, from about 152 mg to about 460 mg, from about 154 mg to about 458 mg, from about 156 mg to about 456 mg, from about 158 mg to about 454 mg, from about 160 mg to about 452 mg, from about 162 mg to about 450 mg, from about 164 mg to about 448 mg, from about 166 mg to about 446 mg, from about 168 mg to about 444 mg, from about 170 mg to about 442 mg, from about 172 mg to about 440 mg, from about 174 to about 438 mg, from about 176 mg to about 436 mg, from about 178 mg to about 434 mg, from about 180 to about 432 mg, from about 182 mg to about 430 mg, from about 184 mg to about 428 mg, from about 186 mg to about 426 mg, from about 188 mg to about 424 mg, from about 190 mg to about 422 mg, from about 192 mg to about 420 mg, from about 194 mg to about 418 mg, from about 196 mg to about 416 mg, from about 198 mg to about 414 mg, from about 200 mg to about 412 mg, from about 202 mg to about 410 mg, from about 204 mg to about 408 mg, from about 206 mg to about 406 mg, from about 208 mg to about 404 mg, from about 210 mg to about 402 mg, from about 212 mg to about 400 mg, from about 214 mg to about 398 mg, from about 216 mg to about 396 mg, from about 218 mg to about 394 mg, from about 220 mg to about 392 mg, from about 222 mg to about 390 mg, from about 224 mg to about 388 mg, from about 226 mg to about 386 mg, from about 228 mg to about 384 mg, from about 230 mg to about 382 mg, from about 232 mg to about 380 mg, from about 234 mg to about 378 mg, from about 236 mg to about 376 mg, from about 238 mg to about 374 mg, from about 240 mg to about 372 mg, from about 242 mg to about 370 mg, from about 244 mg to about 368 mg, from about 246 mg to about 366 mg, from about 248 mg to about 364 mg, from about 250 mg to about 362 mg, from about 252 mg to about 360 mg, from about 254 mg to about 358 mg, from about 256 mg to about 356 mg, from about 258 mg to about 354 mg, from about 260 mg to about 352 mg, from about 262 gm to about 350 mg, from about 264 mg to about 348 mg, from about 266 mg to about 346 mg, from about 268 mg to about 344 mg, from about 270 mg to about 342 mg, from about 272 mg to about 340 mg, from about 274 mg to about 338 mg, from about 276 mg to about 336 mg, from about 278 mg to about 334 mg, from about 280 mg to about 332 mg, from about 282 mg to about 330 mg, from about 284 mg to about 328 mg, from about 286 mg to about 326 mg, from about 288 mg to about 324 mg, from about 290 mg to about 322 mg, from about 292 mg to about 320 mg, from about 294 mg to about 318 mg, from about 296 mg to about 316 mg, from about 298 mg to about 314 mg, from about 300 mg to about 312 mg, 302 mg to about 310 mg, from about 304 mg to about 308 mg, or from about 306 mg to about 310 mg. In other instances, the dose can be from about 4 mg to about 8 mg, from about 8 mg to about 12 mg, from about 12 mg to about 16 mg, from about 16 mg to about 20 mg, from about 20 mg to about 24 mg, from about 24 mg to about 28 mg, from about 28 mg to about 32 mg, from about 32 mg to about 36 mg, from about 36 mg to about 40 mg, from about 40 mg to about 44 mg, from about 44 mg to about 48 mg, from about 48 mg to about 52 mg, from about 52 mg to about 56 mg, from about 56 mg to about 60 mg, from about 60 mg to about 64 mg, from about 64 mg to about 68 mg, from about 68 mg to about 72 mg, from about 72 mg to about 76 mg, from about 76 mg to about 80 mg, from about 80 mg to about 84 mg, from about 84 mg to about 88 mg, from about 88 mg to about 92 mg, from about 92 mg to about 96 mg, from about 96 mg to about 100 mg, from about 100 mg to about 104 mg, from about 104 mg to about 108 mg, from about 108 mg to about 112 mg, from about 112 mg to about 116 mg, from about 116 mg to about 120 mg, from about 120 mg to about 124 mg, from about 124 mg to about 128 mg, from about 128 mg to about 132 mg, from about 132 mg to about 136 mg, from about 136 mg to about 140 mg, from about 140 mg to about 144 mg, from about 144 mg to about 148 mg, from about 148 mg to about 152 mg, from about 152 mg to about 156 mg, from about 156 mg to about 160 mg, from about 160 mg to about 164 mg, from about 164 mg to about 168 mg, from about 168 mg to about 172 mg, from about 172 mg to about 176 mg, from about 176 mg to about 180 mg, from about 180 mg to about 184 mg, from about 184 mg to about 188 mg, from about 188 mg to about 192 mg, from about 192 mg to about 196 mg, from about 196 mg to about 200 mg, from about 200 mg to about 204 mg, from about 204 mg to about 208 mg, from about 208 mg to about 212 mg, from about 212 mg to about 216 mg, from about 216 mg to about 220 mg, from about 220 mg to about 224 mg, from about 224 mg to about 228 mg, from about 228 mg to about 232 mg, from about 232 mg to about 236 mg, from about 236 mg to about 240 mg, from about 240 mg to about 244 mg, from about 244 mg to about 248 mg from about 248 mg to about 252 mg, from about 252 mg to about 256 mg, from about 256 mg to about 260 mg, from about 260 mg to about 264 mg, from about 264 mg to about 268 mg, from about 268 mg to about 272 mg, from about 272 mg to about 276 mg, from about 276 mg to about 280 mg, from about 280 mg to about 284 mg, from about 284 mg to about 288 mg, from about 288 mg to about 292 mg, from about 292 mg to about 296 mg, from about 296 mg to about 300 mg, from about 300 mg to about 304 mg, from about 304 mg to about 308 mg, from about 308 mg to about 312 mg, from about 312 mg to about 316 mg, from about 316 mg to about 320 mg, from about 320 mg to about 324 mg, from about 324 mg to about 328 mg, from about 328 mg to about 332 mg, from about 332 mg to about 336 mg, from about 336 mg to about 340 mg, from about 340 mg to about 344 mg, from about 344 mg to about 348 mg, from about 348 mg to about 352 mg, from about 352 mg to about 356 mg, from about 356 mg to about 360 mg, from about 360 mg to about 364 mg, from about 364 mg to about 368 mg, from about 368 mg to about 372 mg, from about 372 mg to about 376 mg, from about 376 mg to about 380 mg, from about 380 mg to about 384 mg, from about 384 mg to about 388 mg, from about 388 mg to about 392 mg, from about 392 mg to about 396 mg, from about 396 mg to about 400 mg, from about 400 mg to about 404 mg, from about 404 mg to about 408 mg, from about 408 mg to about 412 mg, from about 412 mg to about 416 mg, from about 416 mg to about 420 mg, from about 420 mg to about 424 mg, from about 424 mg to about 428 mg, from about 428 mg to about 432 mg, from about 432 mg to about 436 mg, from about 436 mg to about 440 mg, from about 440 mg to about 444 mg, from about 444 mg to about 448 mg, from about 448 mg to about 452 mg, from about 452 mg to about 456 mg, from about 456 mg to about 460 mg, from about 460 mg to about 464 mg, from about 464 mg to about 468 mg, from about 468 mg to about 472 mg, from about 472 mg to about 476 mg, from about 476 mg to about 480 mg, from about 480 mg to about 484 mg, from about 484 mg to about 488 mg, from about 488 mg to about 492 mg, from about 492 mg to about 496 mg, from about 496 mg to about 500 mg, from about 500 mg to about 504 mg, from about 504 mg to about 508 mg, from about 508 mg to about 512 mg, from about 512 mg to about 516 mg, from about 516 mg to about 520 mg, from about 520 mg to about 524 mg, from about 524 mg to about 528 mg, from about 528 mg to about 532 mg, from about 532 mg to about 536 mg, from about 536 mg to about 540 mg, from about 540 mg to about 544 mg, from about 544 mg to about 548 mg, from about 548 mg to about 552 mg, from about 552 mg to about 556 mg, from about 556 mg to about 560 mg, from about 560 mg to about 564 mg, from about 564 mg to about 568 mg, from about 568 mg to about 572 mg, from about 572 mg to about 576 mg, from about 576 mg to about 580 mg, from about 580 mg to about 584 mg, from about 584 mg to about 588 mg, from about 588 mg to about 592 mg, from about 592 mg to about 596 mg, from about 596 mg to about 600 mg, from about 600 mg to about 604 mg, or from about 604 mg to about 608 mg. In certain instances, the dose can be about 4 mg, about 8 mg, about 12 mg, about 16 mg, about 20 mg, about 24 mg, about 28 mg, about 32 mg, about 36 mg, about 40 mg, about 44 mg, about 48 mg, about 52 mg, about 56 mg, about 60 mg, about 64 mg, about 68 mg, about 72 mg, about 76 mg, about 80 mg, about 84 mg, about 88 mg, about 92 mg, about 96 mg, about 100 mg, about 104 mg, about 108 mg, about 112 mg, about 116 mg, about 120 mg, about 124 mg, about 128 mg, about 132 mg, about 136 mg, about 140 mg, about 144 mg, about 148 mg, about 152 mg, about 156 mg, about 160 mg, about 164 mg, about 168 mg, about 172 mg, about 176 mg, about 180 mg, about 184 mg, about 188 mg, about 192 mg, about 196 mg, about 200 mg, about 204 mg, about 208 mg, about 212 mg, about 216 mg, about 220 mg, about 224 mg, about 228 mg, about 232 mg, about 236 mg, about 240 mg, about 244 mg, about 248 mg, about 252 mg, about 256 mg, about 260 mg, about 264 mg, about 268 mg, about 272 mg, about 276 mg, about 280 mg, about 284 mg, about 288 mg, about 292 mg, about 286 mg, about 300 mg, about 304 mg, about 308 mg, about 312 mg, about 316 mg, about 320 mg, about 324 mg, about 328 mg, about 332 mg, about 336 mg, about 340 mg, about 344 mg, about 348 mg, about 352 mg, about 356 mg, about 360 mg, about 364 mg, about 368 mg, about 372 mg, about 376 mg, about 380 mg, about 384 mg, about 388 mg, about 392 mg, about 396 mg, about 400 mg, about 404 mg, about 408 mg, about 412 mg, about 416 mg, about 420 mg, about 424 mg, about 428 mg, about 432 mg, about 436 mg, about 440 mg, about 444 mg, about 448 mg, about 452 mg, about 456 mg, about 460 mg, about 464 mg, about 468 mg, about 472 mg, about 476 mg, about 480 mg, about 484 mg, about 488 mg, about 492 mg, about 496 mg, about 500 mg, about 504 mg, about 508 mg, about 512 mg, about 516 mg, about 520 mg, about 524 mg, about 528 mg, about 532 mg, about 536 mg, about 540 mg, about 544 mg, about 548 mg, about 552 mg, about 556 mg, about 560 mg, about 564 mg, about 568 mg, about 572 mg, about 576 mg, about 580 mg, about 584 mg, about 588 mg, about 592 mg, about 596 mg, about 600 mg, about 604 mg or about 608 mg. In other certain instances, the dose can be about 4 mg, about 12 mg, about 16 mg, about 32 mg, about 64 mg, about 96 mg, about 128 mg, about 160 mg, about 192 mg, about 224 mg, about 256 mg, about 288 mg, about 304 mg, about 320 mg, about 352 mg, about 384 mg, about 400 mg, about 416 mg, about 448 mg, about 480 mg, about 512 mg, about 544 mg, about 576 mg or about 608 mg, especially about 4 mg, about 12 mg, about 16 mg, about 32 mg, about 96 mg, about 304 mg, about 400 mg or about 608 mg.

Alternatively, the dose can be from about 1 mg to about 5 mg, about 5 mg to about 10 mg, about 10 mg to about 15 mg, about 15 mg to about 20 mg, about 20 mg to about 25 mg, about 25 mg to about 30 mg, about 30 mg to about 35 mg, about 35 mg to about 40 mg, about 40 mg to about 45 mg, about 45 mg to about 50 mg, about 50 mg to about 55 mg, about 55 mg to about 60 mg, about 60 mg to about 65 mg, about 65 mg to about 70 mg, about 70 mg to about 75 mg, about 75 mg to about 80 mg, about 80 mg to about 85 mg, about 85 mg to about 90 mg, about 90 mg to about 95 mg, about 95 mg to about 100 mg, about 100 mg to about 105 mg, about 105 mg to about 110 mg, about 110 mg to about 115 mg, about 115 mg to about 120 mg, about 120 mg to about 125 mg, about 125 mg to about 130 mg, about 130 mg to about 135 mg, about 135 mg to about 140 mg, about 140 mg to about 145 mg, about 145 mg to about 150 mg, about 150 mg to about 155 mg, about 155 mg to about 160 mg, about 160 mg to about 165 mg, about 165 mg to about 170 mg, about 170 mg to about 175 mg, about 175 mg to about 180 mg, about 180 mg to about 185 mg, about 185 mg to about 190 mg, about 190 mg to about 195 mg, about 195 mg to about 200 mg, about 200 mg to about 205 mg, about 205 mg to about 210 mg, about 210 mg to about 215 mg, about 215 mg to about 220 mg, about 220 mg to about 225 mg, about 225 mg to about 230 mg, about 230 mg to about 235 mg, about 235 mg to about 240 mg, about 240 mg to about 245 mg, about 245 mg to about 250 mg, about 250 mg to about 255 mg, about 255 mg to about 260 mg, about 260 mg to about 265 mg, about 265 mg to about 270 mg, about 270 mg to about 275 mg, about 275 mg to about 280 mg, about 280 mg to about 285 mg, about 285 mg to about 290 mg, about 290 mg to about 295 mg, about 295 mg to about 300 mg, about 300 mg to about 305 mg, about 305 mg to about 310 mg, about 310 mg to about 315 mg, about 315 mg to about 320 mg, about 320 mg to about 325 mg, about 325 mg to about 330 mg, about 330 mg to about 335 mg, about 335 mg to about 340 mg, about 340 mg to about 345 mg, about 345 mg to about 350 mg, about 350 mg to about 355 mg, about 355 mg to about 360 mg, about 360 mg to about 365 mg, about 365 mg to about 370 mg, about 370 mg to about 375 mg, about 375 mg to about 380 mg, about 380 mg to about 385 mg, about 385 mg to about 390 mg, about 390 mg to about 395 mg, about 395 mg to about 400 mg, about 400 mg to about 405 mg, about 405 mg to about 410 mg, about 410 mg to about 415 mg, about 415 mg to about 420 mg, about 420 mg to about 425 mg, about 425 mg to about 430 mg, about 430 mg to about 435 mg, about 435 mg to about 440 mg, about 440 mg to about 445 mg, about 445 mg to about 450 mg, about 450 mg to about 455 mg, about 455 mg to about 460 mg, about 460 mg to about 465 mg, about 465 mg to about 470 mg, about 470 mg to about 475 mg, about 475 mg to about 480 mg, about 480 mg to about 485 mg, about 485 mg to about 490 mg, about 490 mg to about 495 mg, about 495 mg to about 500 mg, about 500 mg to about 505 mg, about 505 mg to about 510 mg, about 510 mg to about 515 mg, about 515 mg to about 520 mg, about 520 mg to about 525 mg, about 525 mg to about 530 mg, about 530 mg to about 535 mg, about 535 mg to about 540 mg, about 540 mg to about 545 mg, about 545 mg to about 550 mg, about 550 mg to about 555 mg, about 555 mg to about 560 mg, about 560 mg to about 565 mg, about 565 mg to about 570 mg, about 570 mg to about 575 mg, about 575 mg to about 580 mg, about 580 mg to about 585 mg, about 585 mg to about 590 mg, about 590 mg to about 595 mg, or about 595 mg to about 600 mg.

Alternatively, the dose can be from about 1 mg to about 20 mg, about 10 mg to about 20 mg, about 20 mg to about 30 mg, about 30 mg to about 40 mg, about 40 mg to about 50 mg, about 50 mg to about 60 mg, about 60 mg to about 70 mg, about 70 mg to about 80 mg, about 80 mg to about 90 mg, about 90 mg to about 100 mg, about 100 mg to about 110 mg, about 110 mg to about 120 mg, about 120 mg to about 130 mg, about 130 mg to about 140 mg, about 140 mg to about 150 mg, about 150 mg to about 160 mg, about 160 mg to about 170 mg, about 170 mg to about 180 mg, about 180 mg to about 190 mg, about 190 mg to about 200 mg, about 200 mg to about 210 mg, about 210 mg to about 220 mg, about 220 mg to about 230 mg, about 230 mg to about 240 mg, about 240 mg to about 250 mg, about 250 mg to about 260 mg, about 260 mg to about 270 mg, about 270 mg to about 280 mg, about 280 mg to about 290 mg, about 290 mg to about 300 mg, about 300 mg to about 310 mg, about 310 mg to about 320 mg, about 320 mg to about 330 mg, about 330 mg to about 340 mg, about 340 mg to about 350 mg, about 350 mg to about 360 mg, about 360 mg to about 370 mg, about 370 mg to about 380 mg about 380 mg to about 390 mg, about 390 mg to about 400 mg, about 400 mg to about 410 mg, about 410 mg to about 420 mg, about 420 mg to about 430 mg, about 430 mg to about 440 mg, about 440 mg to about 450 mg, about 450 mg to about 460 mg, about 460 mg to about 470 mg, about 470 mg to about 480 mg, about 480 mg to about 490 mg, about 490 mg to about 500 mg, about 500 mg to about 510 mg, about 510 mg to about 520 mg, about 520 mg to about 530 mg, about 530 mg to about 540 mg, about 540 mg to about 550 mg, about 550 mg to about 560 mg, about 560 mg about 570 mg, about 570 mg to about 580 mg, about 580 mg to about 590 mg, about 590 mg about 600 mg.

Alternatively, the dose can be from about 10 mg to about 25 mg, about 25 mg to about 50 mg, about 50 mg to about 75 mg, about 75 mg to about 100 mg, about 100 mg to about 125 mg, about 125 mg to about 150 mg, about 150 mg to about 175 mg, about 175 mg to about 200 mg, about 200 mg to about 225 mg, about 225 mg to about 250 mg, about 250 mg to about 275 mg, about 275 mg to about 300 mg, about 300 mg to about 325 mg, about 325 mg to about 350 mg, about 350 mg to about 375 mg, about 375 mg to about 400 mg, about 400 mg to about 425 mg, about 425 mg to about 450 mg, about 450 mg to about 475 mg, about 475 mg to about 500 mg, about 500 mg to about 525 mg, about 525 mg to about 550 mg, about 550 mg to about 575 mg, or about 575 mg to about 600 mg.

Alternatively, the dose can be from about 1 mg to about 50 mg, about 50 mg to about 100 mg, about 100 mg to about 150 mg, about 150 mg to about 200 mg, about 200 mg to about 250 mg, about 250 mg to about 300 mg, about 300 mg to about 350 mg, about 350 mg to about 400 mg, about 400 mg to about 450 mg, about 450 mg to about 500 mg, about 500 mg to about 550 mg, or about 550 to about 600 mg.

Alternatively, the dose can be from about 1 mg to about 75 mg, about 75 mg to about 150 mg, about 150 mg to about 225 mg, about 225 mg to about 300 mg, about 300 mg to about 375 mg, about 375 mg to about 450 mg, about 450 mg to about 525 mg, or about 525 mg to about 600 mg.

Alternatively, the dose can be from about 1 mg to about 100 mg, about 100 mg to about 200 mg, about 200 mg to about 300 mg, about 300 mg to about 400 mg, about 400 mg to about 500 mg, or about 500 mg to about 600 mg.

In some instances, the ds RNAi agent includes a sense strand having a nucleotide sequence of SEQ ID NO:1 and includes an antisense strand having a nucleotide sequence of SEQ ID NO:2. In other instances, the sense strand is a nucleotide sequence of SEQ ID NO:3 and the antisense strand is a nucleotide sequence of SEQ ID NO:4.

To facilitate in administering the doses of the ds RNAi agent to an individual in need thereof, the doses can be incorporated into pharmaceutical compositions such as formulations. Such pharmaceutical compositions can include a concentration of the ds RNAi agent or a pharmaceutically acceptable salt thereof (e.g., calcium, magnesium or sodium) in H₂O, especially water-for-injection (WFI).

In some instances, the ds RNAi agent includes a sense strand having a nucleotide sequence of SEQ ID NO:1 and an antisense strand having a nucleotide sequence of SEQ ID NO:2. In other instances, the sense strand is a nucleotide sequence of SEQ ID NO:3 and the antisense strand is a nucleotide sequence of SEQ ID NO:4.

In some instances, the concentration of the ds RNAi agent in the pharmaceutical composition can be from about 100 mg/mL to about 300 mg/mL. In other instances, the concentration can be from about 110 mg/mL to about 290 mg/mL, from about 120 mg/mL to about 280 mg/mL, from about 130 mg/mL to about 270 mg/mL, from about 140 mg/mL to about 260 mg/mL, from about 150 mg/mL to about 250 mg/mL, from about 160 mg/mL to about 240 mg/mL, from about 170 mg. to about 230 mg/mL, from about 180 mg/mL to about 220 mg/mL, from about 190 mg/mL to about 210 mg/mL or about 200 mg/mL. In yet other instances, the concentration can be from about 100 mg/mL to about 110 mg/mL, from about 110 mg/mL to about 120 mg/mL, from about 120 mg/mL to about 130 mg/mL, from about 130 mg/mL to about 140 mg/mL, from about 140 mg/mL to about 150 mg/mL, from about 150 mg/mL to about 160 mg/mL, from about 160 mg/mL to about 170 mg/mL, from about 170 mg/mL to about 180 mg/mL, from about 180 mg/mL to about 190 mg/mL, from about 190 mg/mL to about 200 mg/mL, from about 200 mg/mL to about 210 mg/mL, from about 210 mg/mL to about 220 mg/mL, from about 220 mg/mL to about 230 mg/mL, from about 230 mg/mL to about 240 mg/mL, from about 240 mg/mL to about 250 mg/mL, from about 250 mg/mL to about 260 mg/mL, from about 260 mg/mL to about 270 mg/mL, from about 270 mg/mL to about 280 mg/mL, from about 280 mg/mL to about 290 mg/mL, or from about 290 mg/mL to about 300 mg/mL. In certain instances, the concentration can be about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, about 200 mg/mL, about 210 mg/mL, about 220 mg/mL, about 230 mg/mL, about 240 mg/mL, about 250 mg/mL, about 260 mg/mL, about 270 mg/mL, about 280 mg/mL, about 290 mg/mL or about 300 mg/mL, especially about 160 mg/mL or about 200 mg/mL.

In some instances, the pH of the pharmaceutical compositions can be from about 6.0 to about 8.0. In other instances, the pH can be from about 6.1 to about 7.9, from about 6.2 to about 7.8, from about 6.3 to about 7.7, from about 6.4 to about 7.6, from about 6.5 to about 7.5, from about 6.6 to about 7.4, from about 6.7 to about 7.3, from about 6.8 to about 7.2, from about 6.9 to about 7.1, or about 7.0. In yet other instances, the pH can be from about from about 6.0 to about 6.1, from about 6.1 to about 6.2, from about 6.2 to about 6.3, from about 6.3 to about 6.4, from about 6.4 to about 6.5, from about 6.5 to about 6.6, from about 6.6 to about 6.7, from about 6.7 to about 6.8, from about 6.8 to about 6.9, from about 6.9 to about 7.0, from about 7.0 to about 7.1, from about 7.1 to about 7.2, from about 7.2 to about 7.3, from about 7.3 to about 7.4, from about 7.4 to about 7.5, from about 7.5 to about 7.6, from about 7.6 to about 7.7, from about 7.7 to about 7.8, from about 7.8 to about 7.9, from about 7.9 to about 8.0. In certain instances, the pH can be about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9 or about 8.0, especially about 7.0. Sodium hydroxide (NaOH) or hydrogen chloride (HCl) can be used to adjust the pH as may be needed.

In some instances, the pharmaceutical compositions are sterile when first produced. As such, they optionally can include a preservative that is compatible with the other components of the composition and that may be added at sufficient strength to meet applicable regulatory anti-microbial preservative requirements. Pharmaceutically acceptable preservatives are known to one of skill in the art (see, e.g., Remington: The Science and Practice of Pharmacy (Troy, Ed., 21^{st} Edition, Lippincott, Williams & Wilkins, 2006). In other instances, the formulations herein are preservative free.

In some instances, the pharmaceutical compositions can be stored at a temperature from about 2° C. to about 8° C. In other instances, the temperature can be from about 3° C. to about 7° C., from about 4° C. to about 6° C., or about 5° C. In yet other instances, the temperature can be from about 2° C. to about 3° C., from about 3° C. to about 4° C., from about 4° C. to about 5° C., from about 5° C. to about 6° C., from about 6° C. to about 7° C., or from about 7° C. to about 8° C. In certain instances, the temperature can be about 2° C., about 3° C., about 4° C., about 5° C., about 6° C., about 7° C. or about 8° C. In other instances, the formulations can be stored at a temperature from about 20° C. to about 22° C. (i.e., room temperature) or higher. For example, the pharmaceutical compositions can be stored at a temperature from about 30° C. to about 40° C. In yet other instances, the temperature can be from about 31° C. to about 39° C., from about 32° C. to about 38° C., from about 33° C. to about 37° C., from about 34° C. to about 36° C. or about 35° C. Alternatively, the temperature can be about 30° C., about 31° C., about 32° C., about 33° C., about 34° C., about 35° C., about 36° C., about 37° C., about 38° C., about 39° C. or about 40° C.

In some instances, such as for low doses, the pharmaceutical compositions can be diluted with about 0.9% NaCl for injection. In other instances, the pharmaceutical compositions can be diluted with mannitol or dextrose for injection.

In one particular instance, a pharmaceutical composition includes a ds RNAi agent that modulates LPA expression or pharmaceutically acceptable salt thereof having a sense strand with a nucleotide sequence of SEQ ID NO:1 and an antisense strand with a nucleotide sequence of SEQ ID NO:2 in H₂O at pH 7.0, where the ds RNAi agent is at a concentration from about 150 mg/mL to about 170 mg/mL, especially about 160 mg/mL.

In another particular instance, a pharmaceutical composition includes a ds RNAi agent that modulates LPA expression or a pharmaceutically acceptable salt thereof having a sense strand with a nucleotide sequence of SEQ ID NO:1 and an antisense strand with a nucleotide sequence of SEQ ID NO:2 in H₂O at pH 7.0, where the ds RNAi agent is at a concentration from about 190 mg/mL to about 210 mg/mL, especially about 200 mg/mL.

In another particular instance, a pharmaceutical composition includes a ds RNAi agent that modulates LPA expression or a pharmaceutically acceptable salt thereof having a sense strand with a nucleotide sequence of SEQ ID NO:3 and an antisense strand with a nucleotide sequence of SEQ ID NO:4 in H₂O at pH 7.0, where the ds RNAi agent is at a concentration from about 150 mg/mL to about 170 mg/mL, especially about 160 mg/mL.

In another particular instance, a pharmaceutical composition includes a ds RNAi agent that modulates LPA expression or a pharmaceutically acceptable salt thereof having a sense strand with a nucleotide sequence of SEQ ID NO:3 and an antisense strand with a nucleotide sequence of SEQ ID NO:4 in H₂O at pH 7.0, where the ds RNAi agent is at a concentration from about 190 mg/mL to about 210 mg/mL, especially about 200 mg/mL.

The pharmaceutical compositions can be administered IV or SC, especially SC. The pharmaceutical compositions can be administered using a pre-filled, disposable pen, reusable pen or automatic pen injector. Alternatively, the pharmaceutical compositions may be administered using a single-use vial, a multi-use vial or a pump device. In some instances, the device is an automatic injection apparatus as described in U.S. Pat. No. 8,734,394.

The pharmaceutical compositions therefore may be presented in a pre-filled syringe/multi-use vial. Such pre-filled syringe/multi-use vial may be useful for administering from about 0.5 mL to about 5 mL of the formulation per individual per dose. The dose may be administered using a dosing schedule determined by a clinician, physician or other trained medical professional.

Alternatively, the pharmaceutical compositions can be prepared for a cartridge and therefore may differ from the above by including an optional preservative, especially if for multi-use.

Alternatively, the pharmaceutical compositions can be prepared as part of an article of manufacture comprising the ds RNAi agent, where the article of manufacture can be a multi-use vial, a reusable pen injector, a pre-filled, disposable pen, an autoinjector or a pump.

In view of the above, the pharmaceutical compositions are associated with acceptable shelf-life stability, in-use stability and acceptable injection site experience.

### Dosing Regimens and Other Methods

The ds RNAi agent that modulates LPA and compositions thereof such as the doses and formulations herein can be used for attenuating, preventing and/or treating diseases, disorders and/or conditions associated with LPA expression. For example, the ds RNAi agent or compositions including the same may be used in individuals to reduce the risk of MACE in individuals with or at high risk of ASCVD and/or high Lp(a) levels or may be used in individuals at risk for a first CV event to reduce such risk.

The methods can include the steps described herein, and these maybe be, but not necessarily, carried out in the sequence as described. Other sequences, however, also are conceivable. Moreover, individual or multiple steps may be carried out either in parallel and/or overlapping in time and/or individually or in multiply repeated steps. Furthermore, the methods may include additional, unspecified steps.

While the details of various methods are provided below, each can include an optional step of selecting an individual who has or is predisposed to having a disease, disorder and/or condition associated with LPA expression. For example, and in some instances, the individual is an adult with elevated Lp(a) and established CV disease. In some instances, the individual is an adult at risk for a first CV event and optionally may have elevated Lp(a) level. In other instances, the individual can have a Lp(a) level of at least about ≥75 nmol/L prior to administering a dose. In yet other instances, the individual has a Lp(a) at least about 100 nmol/L, about 125 nmol/L, about 150 nm/L, about 175 nmol/L or about 200 nmol/L prior to administering a dose. In yet other instances, the individual has a SNP of LPA selected from rs10455872 and rs3798220. In some instances, the disease, disorder and/or condition associated with LPA expression includes, but is not limited to, atherosclerosis, CAVS, cardiometabolic diseases, cardiovascular events including coronary revascularization, CV mortality, cerebrovascular disease, chronic kidney disease, coronary heart disease, dyslipidemia, heart failure, MI and stroke, and/or peripheral vascular disease, especially atherosclerosis, cardiometabolic diseases and dyslipidemia.

For example, methods are provided for reducing LPA expression in an individual, where such methods include a step of administering to the individual a first dose of a ds RNAi agent that modulates LPA expression or a pharmaceutical composition thereof such as a formulation herein, where the ds RNAi agent includes a sense strand having a nucleotide sequence of SEQ ID NO:1 or 3 and an antisense strand having a nucleotide sequence of SEQ ID NO:2 or 4, where the first dose can be from about 4 mg to about 608 mg.

In some instances, the first dose can be administered IV or can be administered SC, especially SC.

In some instances, the sense strand is SEQ ID NO:3. In other instances, the antisense strand is SEQ ID NO: 4. In certain instances, the sense strand is SEQ ID NO:3 and the antisense strand is SEQ ID NO:4.

In some instances, the first dose can be about 4 mg, about 12 mg, about 16 mg, about 32 mg, about 64 mg, about 96 mg, about 128 mg, about 160 mg, about 192 mg, about 224 mg, about 256 mg, about 288 mg, about 304 mg, about 320 mg, about 352 mg, about 384 mg, about 400 mg, about 416 mg, about 448 mg, about 480 mg, about 512 mg, about 544 mg, about 576 mg or about 608 mg. In other instances, the first dose can be about 4 mg, about 12 mg, about 16 mg, about 32 mg, about 96 mg, about 304 mg, about 400 mg or about 608 mg.

The method also can include a step of administering to the individual one or more subsequent doses (i.e., at least a second dose) of the ds RNAi agent, where the one or more subsequent doses can be from about 4 mg to about 608 mg, and where the one or more subsequent doses can be the same as the first dose or can be different from the first dose and can be administered at about Q1M, about Q3M, about Q6M, about Q9M or about Q12M after the first dose or a previous dose. Alternatively, the one or more subsequent doses can be administered at about two months (Q2M), about four months (Q4M), about five months (Q5M), about seven months (Q7M), about eight months (Q8M), about ten months (Q10M) or about eleven months (Q11M) after the first dose or a previous dose.

In some instances, the one or more subsequent doses can be administered IV or can be administered SC, especially SC.

In some instances, the one or more subsequent doses can be about 4 mg, about 12 mg, about 16 mg, about 32 mg, about 64 mg, about 96 mg, about 128 mg, about 160 mg, about 192 mg, about 224 mg, about 256 mg, about 288 mg, about 304 mg, about 320 mg, about 352 mg, about 384 mg, about 400 mg, about 416 mg, about 448 mg, about 480 mg, about 512 mg, about 544 mg, about 576 mg or about 608 mg. In other instances, the one or more subsequent doses can be about 4 mg, about 12 mg, about 16 mg, about 32 mg, about 96 mg, about 304 mg, about 400 mg or about 608 mg.

When a subsequent dose (i.e., third, fourth, fifth, et seq. dose) is administered, the subsequent dose can be the same as a second dose (or from any preceding dose in the case of a fourth, fifth, et seq. dose) or can be different from the second dose (or from any preceding dose in the case of a fourth, fifth, et seq. dose). In addition, the subsequent dose can be administered from about Q1M to about Q12M (i.e., about Q1M, Q2M, Q3M, Q4M, Q5M, Q6M, Q7M, Q8M, Q9M, Q10M, Q11M or Q12M) from the second dose (or from any preceding dose in the case of a fourth, fifth, et seq. dose).

In some instances, the one or more subsequent doses can be about 4 mg administered every Q1M, about 4 mg administered every Q3M, about 4 mg administered every Q6M, about 4 mg administered every Q9M or about 4 mg administered about every Q12M.

In some instances, the one or more subsequent doses can be about 12 mg administered every Q1M, about 12 mg administered about every Q3M, about 12 mg administered about every Q6M, about 12 mg administered about every Q9M or about 12 mg administered about every Q12M.

In some instances, the one or more subsequent doses can be about 16 mg administered about every Q1M, about 16 mg administered about every Q3M, about 16 mg administered about every Q6M, about 16 mg administered about every Q9M or about 16 mg administered about every Q12M.

In some instances, the one or more subsequent doses can be about 32 mg administered about every Q1M, about 32 mg administered about every Q3M, about 32 mg administered about every Q6M, about 32 mg administered about every Q9M or about 32 mg administered about every Q12M.

In some instances, the one or more subsequent doses can be about 96 mg administered about every Q1M, about 96 mg administered about every Q3M, about 96 mg administered about every Q6M, about 96 mg administered about every Q9M or about 96 mg administered about every Q12M.

In some instances, the one or more subsequent doses can be about 304 mg administered about every Q1M, about 304 mg administered about every Q3M, about 304 mg administered about every Q6M, about 304 mg administered about every Q9M or about 304 mg administered about every Q12M.

In some instances, the one or more subsequent doses can be about 400 mg administered about every Q1M, about 400 mg administered about every Q3M, about 400 mg administered about every Q6M, about 400 mg administered about every Q9M or about 400 mg administered about every Q12M.

In some instances, the one or more subsequent doses can be about 608 mg administered about every Q1M, about 608 mg administered about every Q3M, about 608 mg administered about every Q6M, about 608 mg administered every Q9M or about 608 mg administered every Q12M.

In other instances, the one or more subsequent doses can be about 400 mg, where at least one of the subsequent doses can be administered about every Q6M, and where other subsequent doses can be administered about every Q12M.

Assuming a pharmaceutical formulation with a concentration of ds RNAi agent at about 160 mg/mL is used, exemplary volumes to deliver the doses note above are as follows:
about 25 μL to deliver the about 4 mg dose,
about 75 μL to deliver the about 12 mg dose,
about 0.1 mL to deliver the about 16 mg dose,
about 0.2 mL to deliver the about 32 mg dose,
about 0.6 mL to deliver the about 96 mg dose,
about 1.9 mL to deliver the about 304 mg dose,
about 2.5 mL to deliver the about 400 mg dose, and
about 3.8 mL to deliver the about 608 mg dose.

Alternatively, and a pharmaceutical formulation with a concentration of ds RNAi agent at about 200 mg/mL is used, exemplary volumes to deliver the doses note above are as follows:
about 20 μL to deliver the about 4 mg dose,
about 60 μL to deliver the about 12 mg dose,
about 80 μL to deliver the about 16 mg dose,
about 0.2 mL to deliver the about 32 mg dose,
about 0.5 mL to deliver the about 96 mg dose,
about 1.5 mL to deliver the about 304 mg dose,
about 2.0 mL to deliver the about 400 mg dose, and
about 3.0 mL to deliver the about 608 mg dose.

As noted above, the one or more subsequent doses (e.g., second, third, fourth, fifth, et seq. dose) can be administered at the same dosing frequency or can be administered at a different dosing frequency distinct from one another, which can be guided by, for example, a degree of change in the individual's LPA expression. As such, the methods also can include a step of adjusting the one or more subsequent doses after comparing the individual's LPA expression following a dose to a control or a previously measured/recorded LPA expression.

In addition to the above, methods are provided for attenuating, preventing and/or treating diseases, disorders and/or conditions associated with LPA expression, where such methods include a step of administering to an individual in need thereof a first dose of a ds RNAi agent that modulates LPA expression or a pharmaceutical composition thereof such as a formulation herein, where the ds RNAi agent includes a sense strand having a nucleotide sequence of SEQ ID NO:1 or 3 and an antisense strand having a nucleotide sequence of SEQ ID NO:2 or 4, and where the first dose can be from about 4 mg to about 608 mg.

In some instances, the first dose can be administered IV or can be administered SC, especially SC.

In some instances, the sense strand is SEQ ID NO:3. In other instances, the antisense strand is SEQ ID NO:4. In certain instances, the sense strand is SEQ ID NO:3 and the antisense strand is SEQ ID NO:4.

In some instances, the first dose can be about 4 mg, about 12 mg, about 16 mg, about 32 mg, about 64 mg, about 96 mg, about 128 mg, about 160 mg, about 192 mg, about 224 mg, about 256 mg, about 288 mg, about 304 mg, about 320 mg, about 352 mg, about 384 mg, about 400 mg, about 416 mg, about 448 mg, about 480 mg, about 512 mg, about 544 mg, about 576 mg or about 608 mg.

The method also can include a step of administering to the individual one or more subsequent doses of the ds RNA agent, where the one or more subsequent doses can be from about 4 mg to about 608 mg, and where the one or more subsequent doses can be the same as the first dose or can be different from the first dose and can be administered about Q1M, about Q3M, about Q6M, about Q9M or about Q12M after the first dose. Alternatively, the at least second dose can be administered about Q2M, about Q4M, about Q5M, about Q7M, about Q8M, about Q10M or about Q11M after the first dose.

In some instances, the one or more subsequent doses can be administered IV or can be administered SC, especially SC.

In some instances, the one or more subsequent doses can be about 4 mg, about 12 mg, about 16 mg, about 32 mg, about 64 mg, about 96 mg, about 128 mg, about 160 mg, about 192 mg, about 224 mg, about 256 mg, about 288 mg, about 304 mg, about 320 mg, about 352 mg, about 384 mg, about 400 mg, about 416 mg, about 448 mg, about 480 mg, about 512 mg, about 544 mg, about 576 mg or about 608 mg. In other instances, the one or more subsequent doses can be about 4 mg, about 12 mg, about 16 mg, about 32 mg, about 96 mg, about 304 mg, about 400 mg or about 608 mg.

When a subsequent dose (i.e., third, fourth, fifth, et seq. dose) is administered, the subsequent dose can be the same as a second dose (or from any preceding dose in the case of a fourth, fifth, et seq. dose) or can be different from the second dose (or from any preceding dose in the case of a fourth, fifth, et seq. dose). In addition, the subsequent dose can be administered from about Q1M to about Q12M (i.e., about Q1M, Q2M, Q3M, Q4M, Q5M, Q6M, Q7M, Q8M, Q9M, Q10M, Q11M or Q12M) from the second dose (or from any preceding dose in the case of a fourth, fifth, et seq. dose).

In some instances, the one or more subsequent doses can be about 4 mg administered every Q1M, about 4 mg administered every Q3M, about 4 mg administered every Q6M, about 4 mg administered every Q9M or about 4 mg administered about every Q12M.

In some instances, the one or more subsequent doses can be about 12 mg administered every Q1M, about 12 mg administered about every Q3M, about 12 mg administered about every Q6M, about 12 mg administered about every Q9M or about 12 mg administered about every Q12M.

In some instances, the one or more subsequent doses can be about 16 mg administered about every Q1M, about 16 mg administered about every Q3M, about 16 mg administered about every Q6M, about 16 mg administered about every Q9M or about 16 mg administered about every Q12M.

In some instances, the one or more subsequent doses can be about 32 mg administered about every Q1M, about 32 mg administered about every Q3M, about 32 mg administered about every Q6M, about 32 mg administered about every Q9M or about 32 mg administered about every Q12M.

In some instances, the one or more subsequent doses can be about 96 mg administered about every Q1M, about 96 mg administered about every Q3M, about 96 mg administered about every Q6M, about 96 mg administered about every Q9M or about 96 mg administered about every Q12M.

In some instances, the one or more subsequent doses can be about 304 mg administered about every Q1M, about 304 mg administered about every Q3M, about 304 mg administered about every Q6M, about 304 mg administered about every Q9M or about 304 mg administered about every Q12M.

In some instances, the one or more subsequent doses can be about 400 mg administered about every Q1M, about 400 mg administered about every Q3M, about 400 mg administered about every Q6M, about 400 mg administered about every Q9M or about 400 mg administered about every Q12M.

In some instances, the one or more subsequent doses can be about 608 mg administered about every Q1M, about 608 mg administered about every Q3M, about 608 mg administered about every Q6M, about 608 mg administered every Q9M or about 608 mg administered every Q12M.

In other instances, the one or more subsequent doses can be about 400 mg, where at least one of the subsequent doses can be administered about every Q6M, and where other subsequent doses can be administered about every Q12M.

As noted above, the one or more subsequent doses (e.g., second, third, fourth, fifth, et seq. dose) can be administered at the same dosing frequency or can be administered at a different dosing frequency distinct from one another, which can be guided by, for example, a degree of change in the individual's Lp(a) concentration (or other lipid profile constituent) following a dose. As such, the methods also can include a step of measuring or recording at least one of the individual's lipid profile constituent values such as, for example, Apo(a), ApoB, HDL-C, LDL-C, Lp(a), total cholesterol (TC) and/or triglycerides (TG) and comparing the at least one value to a control value or to another measured/recorded value from the individual. In some instances, the method further can include a step of adjusting the one or more subsequent doses after comparing the measured/recorded value to a control or a previously measured/recorded value of the individual.

In any of the above methods, each dose can be about 4 mg, about 12 mg, about 16 mg, about 32 mg, about 96 mg, about 304 mg, about 400 mg or about 608 mg, where a first dose is administered at day 0 and each subsequent dose (i.e., second, third, fourth, fifth, et seq. dose) is administered at a frequency of about every Q6M therefrom.

In any of the above methods, each dose can be about 4 mg, about 12 mg, about 16 mg, about 32 mg, about 96 mg, about 304 mg, about 400 mg or about 608 mg, where a first dose is administered at day 0, a second dose is administered about Q6M from the first dose, and each subsequent dose (i.e., third, fourth, fifth, et seq. dose) is administered at a frequency of about every Q12M from the second dose. Alternatively, a first dose is administered at day 0, a second dose is administered about Q6M from the first dose, a third dose is administered about Q6M from the second dose and each subsequent dose (i.e., fourth, fifth, et seq. dose) is administered at a frequency of about every Q12M from the third dose. Alternatively, a first dose is administered at day 0, a second dose is administered about Q6M from the first dose, a third dose is administered about Q6M from the second dose, a fourth dose is administered about Q6M from the third dose and each subsequent dose (i.e., fifth, et seq. dose) is administered at a frequency of about every Q12M from the fourth dose.

In any of the above methods, each dose can be about 4 mg, about 12 mg, about 16 mg, about 32 mg, about 96 mg, about 304 mg, about 400 mg or about 608 mg, where a first dose is administered at day 0 and each subsequent dose (i.e., second, third, fourth, fifth, et seq. dose) is administered at a frequency of about every Q12M therefrom.

In any of the above methods, the injections can be administered via a 27-G needle into the SC tissue of the abdominal wall of the individual, about 10 cm from the umbilicus. For example, injections can be administered at an about 450 angle without pinching a skinfold. If more than 2 mL solution is required to deliver a dose, then it can be split into injections up to a maximum volume of 2 mL/injection. In this manner, multiple injections can be performed in quick succession, and each can be delivered in 1 of the 4 quadrants of the anterior abdominal wall. In other instances, a single injection of ≥2.0 mL can be used to deliver the dose.

In any of the above, the method also can include a step of measuring/recording the individual's frequency and severity of AEs following a dose. In some instances, the method further can include a step of adjusting the one or more subsequent doses of the ds RNAi agent after assessing the individual's frequency and severity of AEs.

In any of the above, the individual's Lp(a) level falls to or is maintained at <150 mg/dL, <125 mg/dL, <100 mg/dL, <75 mg/dL or <50 mg/dL following a dose of the ds RNAi agent.

In any of the above, the individual's Lp(a) decreases by about 90% within about 14 days following a dose of the ds RNAi agent.

In any of the above, the individual may be on a concomitant lipid-lowering therapy such as, for example, a therapy to reduce LDL-C levels (e.g., a PCSK9 inhibitor, a statin, a cholesterol absorption inhibitor, LDL apheresis or combinations thereof).

### EXAMPLES

The following non-limiting examples are offered for purposes of illustration, not limitation.

### Pharmaceutical Formulation

### Example 1: Pharmaceutical Formulation Including a Ds RNAi Agent

The formulation was prepared substantially as described herein, where the formulation includes a ds RNAi agent (e.g., sense strand of SEQ ID NO:3 and antisense strand of SEQ ID NO:4; sodium equivalent) at 160 mg/mL or 200 mg/mL in H₂O (WFI). The formulation is supplied for use as a solution formulation in glass vials.

### Chemical and Physical Stability

### Example 2: In-Use Stability Studies

Methods: The formulation was prepared as described in Example 1. Formulation stability was evaluated in glass vials at various temperatures from 2° C.-8° C. and at 25° C. and 40° C., as well as under extreme elevated conditions of 60° C. and 80° C. for various durations. The formulation was monitored for degradation using denaturing and non-denaturing methods.

Results: Based upon these studies, the formulation has extended stability of up to 5 years at 2° C.-8° C. and 25° C., as well as were stable for several months at 40° C.

TABLE 1
Chemical and Physical Stability of GMP ds RNAi Agent DP Formulation.
Formulation
(160 mg/mL in WFI, pH 7)

| | | Initial | 1- | 3- | 6- | 9- | 12- |
|---|---|---|---|---|---|---|---|
| Study | Temp. | (T₀) | Month | Months | Months | Months | Months |
| pH | 5° C. | 7.4 | 7.4 | 7.4 | 7.5 | 7.5 | 7.5 |
| | 25° C. | 7.4 | 7.4 | 7.5 | 7.8 | N/A | 7.6 |
| | 40° C. | 7.4 | 7.6 | 7.6 | 7.6 | N/A | N/A |
| Particulate | 5° C. | 1 | 2 | 1 | 18 | 5 | 1 |
| Matter | | 0 | 0 | 0 | 0 | 0 | 0 |
| (HIAC) | 25° C. | 1 | 4 | 3 | 23 | N/A | 7 |
| >10 μm | | 0 | 0 | 0 | 1 | | 1 |
| >25 μm | 40° C. | 1 | N/A | N/A | 47 | N/A | N/A |
| | | 0 | | | 3 | | |
| Description | 5° C. | Yellow | Yellow | Yellow | Yellow | Yellow | Yellow |
| (visual) | 25° C. | liquid and | liquid | liquid | liquid | liquid | liquid |
| | 40° C. | no visible | and no | and no | and no | and no | and no |
| | | particles | visible | visible | visible | visible | visible |
| | | | particles | particles | particles | particles | particles |

TABLE 2
Chemical Stability of GMP ds RNAi Agent DP
Formulation (concentration and strand purity).
Formulation
(160 mg/mL in WFI, pH 7)

| | | Initial | 1- | 3- | 6- | 9- | 12- |
|---|---|---|---|---|---|---|---|
| Study | Temp. | (T₀) | Month | Months | Months | Months | Months |
| UV | 5° C. | 157 | 155 | 160 | 157 | 151 | 148 |
| Content | 25° C. | 157 | 154 | 161 | 156 | N/A | 153 |
| | 40° C. | 157 | 156 | 162 | 159 | N/A | N/A |
| Denaturing | 5° C. | 38.7 | 38.6 | 38.2 | 38.2 | 39.5 | 37.9 |
| Purity | 25° C. | 38.7 | 38.5 | 38.1 | 38.2 | N/A | 37.9 |
| L02B | 40° C. | 38.7 | 38.6 | 38.1 | 38.1 | N/A | N/A |
| Strand | | | | | | | |
| (%) | | | | | | | |
| Denaturing | 5° C. | 49.2 | 49.2 | 47.9 | 47.8 | 48.8 | 49.0 |
| Purity | 25° C. | 49.2 | 49.2 | 47.9 | 47.8 | N/A | 49.1 |
| L02C | 40° C. | 49.2 | 49.3 | 48 | 49.2 | N/A | N/A |
| Strand | | | | | | | |
| (%) | | | | | | | |
| Non- | 5° C. | 97.3 | 97.8 | 97.7 | 97 | 97.6 | 97.7 |
| Denaturing | 25° C. | 97.3 | 97.8 | 97.8 | 97.1 | N/A | 97.7 |
| Duplex | 40° C. | 97.3 | 97.9 | 97.8 | 97.1 | N/A | N/A |
| Purity | | | | | | | |
| (%) | | | | | | | |

### Dosing

### Example 3: A Study of a ds RNAi Agent for Modulating LPA Expression in Healthy Adults

Methods: Multicenter studies were designed to evaluate the safety, tolerability, pharmacodynamics (PD) and pharmacokinetics (PK) of a ds RNAi agent for modulating LPA expression (SEQ ID NOS:3 and 4 for the sense and antisense strands, respectively) in otherwise healthy individuals with elevated Lp(a) (no CVD with Lp(a)≥75 nmol/L or 30 mg/dL) (Part A) or in healthy individuals (Part B). Such studies were a randomized, investigator- and participant-blind, and placebo-controlled.

In Part A, individuals were SC administered single-ascending doses of the ds RNAi agent at 4 mg, 12 mg, 32 mg, 96 mg, 304 mg or 608 mg. Individuals were randomly assigned to receive either the ds RNAi agent or placebo (0 mg; 0.900 NaCl solution for injection) in a 6:2 ratio. Staggered dosing was performed for each cohort in Part A, in which a new higher dose of the ds RNAi agent was administered.

In Part B, individuals were SC administered a single dose of 304 mg or 608 mg. Individuals were randomly assigned to receive either the ds RNAi agent or placebo (0 mg; 0.9% NaCl solution for injection) in a 1:1:1 ratio.

All injections were administered into the tissue of the abdominal wall. The ds RNAi agent was provided as a 160 mg/mL solution. Placebo was administered as 2.5 mL 0.9% NaCl solution for injection.

Blood samples were collected pre-dose and at 0.5, 1.5, 3, 6, 9, 12, 16, 24, 36, 48 and 72 hrs post-dose and on day 8, 15, 22, 29, 85 and 169 post-dose to determine plasma concentration of the ds RNAi agent. Plasma concentrations were used to compute PD and/or PK parameters.

PD analysis: included Lp(a), ApoB and a lipid panel.

PK analysis: included AUC(₀₋ₜₗₐₛₜ), AUC(_{0-∞}), Cₘₐₓ, tₘₐₓ, t½ and CL/F.

Safety analysis: included physical examinations, vital sign assessments, 12-lead ECG, clinical laboratory assessments, PLG activity, Pal-1, tPA, A2AP, cytokine panel, assessment for injection-site reactions, immunogenicity and adverse events.

Results: No effect was observed on ApoB and lipids up to Day 169.

TABLE 3
Summary of PD Parameters (Lp(a))—Part A.

| | Change in Lp(a) | | | | | | | | | | | | | |
|---|---|---|---|---|---|---|---|---|---|---|---|---|---|---|
| | (%) | | | | | | | | | | | | | |
| Dose | Day | Day | Day | Day | Day | Day | Day | Day | Day | Day | Day | Day | Day | Day |
| (mg) | 1 | 2 | 4 | 8 | 15 | 22 | 29 | 43 | 57 | 71 | 85 | 113 | 141 | 169 |
| 0 | 0 | 11.0 | 22.1 | 4.4 | −1.0 | 0.7 | −0.3 | −1.2 | −2.1 | 2.5 | 7.5 | −6.5 | −4.3 | −4.0 |
| 4 | 0 | 0.1 | −1.0 | −15.2 | −23.2 | −20.0 | −27.1 | −28.6 | −35.3 | −28.1 | −40.1 | −29.0 | −11.6 | −21.1 |
| 12 | 0 | 3.5 | 14.8 | −4.3 | −29.4 | −37.8 | −44.7 | −53.3 | −51.6 | −51.1 | −47.8 | −47.1 | −42.2 | −33.5 |
| 32 | 0 | 4.7 | 20.5 | −7.9 | −37.5 | −52.6 | −67.6 | −69.8 | −75.7 | −71.6 | −71.0 | −72.4 | −65.3 | −60.5 |
| 96 | 0 | 15.8 | 9.9 | −33.7 | −64.2 | −77.8 | −85.7 | −88.2 | −89.4 | −88.8 | −85.9 | −86.9 | −82.1 | −83.2 |
| 304 | 0 | 6.3 | −1.0 | −46.2 | −77.3 | −88.2 | −93.2 | −95.0 | −96.5 | −96.1 | −96.0 | −96.5 | −94.7 | −93.3 |
| 608 | 0 | 4.2 | −6.0 | −58.0 | −83.7 | −93.2 | −96.5 | −97.0 | −97.0 | −97.0 | −97.0 | −97.0 | −97.0 | −97.0 |

TABLE 4
Summary of PK Parameters—Part A (6 individuals/dose).

| | PK Parameter | | | | | |
|---|---|---|---|---|---|---|
| | (Geometric Means) | | | | | |
| Dose | AUC₍₀₋ₜₗₐₛₜ₎ | AUC_{(0-∞)} | Cₘₐₓ | tₘₐₓ | t½ | CL/F |
| (mg) | (ng · h/mL) | (ng · h/mL) | (ng/ml) | (hr) | (hr) | (L/hr) |
| 4 | 101 | NC | 15.3 | 6 | NC | NC |
| 12 | 794 | 1010 | 75.5 | 4.6 | 3.7 | 11.9 |
| 32 | 2950 | 2840 | 139 | 7.5 | 7.3 | 11.3 |
| 96 | 8210 | 8710 | 431 | 9.1 | 6.3 | 11.0 |
| 304 | 37800 | 38600 | 1740 | 10.5 | 5.8 | 7.9 |
| 608 | 63100 | 62500 | 2610 | 9.0 | 7.0 | 9.7 |

Abbreviations:
NC = not calculated

TABLE 5
Summary of PK Parameters—Part B (6 individuals/dose).

| | PK Parameter | | | | | |
|---|---|---|---|---|---|---|
| | Geometric Means | | | | | |
| Dose | AUC₍₀₋ₜₗₐₛₜ₎ | AUC_{(0-∞)} | Cₘₐₓ | tₘₐₓ | t½ | CL/F |
| (mg | (ng · h/mL) | (ng · h/mL) | (ng/mL) | (hr) | (hr) | (L/hr) |
| 304 | 28800 | 29700 | 1220 | 9.0 | 6.8 | 10.2 |
| 608 | 70900 | 72800 | 3290 | 7.5 | 7.2 | 8.4 |

PD: As for PD, single doses (i.e., 4, 12, 32, 96, 304 and 608 mg) of the ds RNAi agent led to significant, dose-dependent and durable reductions in Lp(a). Specifically, Lp(a) levels decreased >90%, and durability was >24 weeks after a single dose. For example, on Day 169, the mean reduction in Lp(a) after a single SC dose of 608 mg of the ds RNAi agent was −96.99% (mean baseline: 122.70 nmol/L). In contrast, the mean reduction in Lp(a) was −4% (mean baseline: 110.35 nmol/L) for the pooled placebo group.

PK: As for PK, it was about dose proportional across the dose range tested. The Cₘₐₓ ranged from about 4.6 hr to about 10.5 hr (median tₘₐₓ) with a mean terminal t_{1/2} of about 4 hr to about 7 hr. Single doses of the ds RNAi agent provided durable Lp(a) lowering thereby allowing for dosing every 6-12 months.

Safety: As for the safety profile, single doses (range of 4 mg to 608 mg) of the ds RNAi agent were safe and well tolerated in healthy individuals with elevated Lp(a) levels. In fact, there were no serious adverse events (AEs) related to the ds RNAi agent or discontinuations due to AEs. The majority of treatment-emergent AEs (TEAEs) were mild in severity, and no apparent dose relationship was observed. The most frequent TEAEs were headache, redness on the skin (ECG patch), COVID-19 infection, fever and runny nose. In addition, no clinically significant changes in pulse rate, blood pressure or ECGs/QTc interval were observed.

Other: There were no treatment-related trends or clinically significant changes in laboratory safety parameters, apart from two individuals who had increases in liver enzymes (i.e., for Part A: 1 individual at the 4 mg dose and 1 individual at the 96 mg dose) and 4 individuals who had increases in CK (i.e., for Part A: 1 individual at the 4 mg dose and 1 individual at the 608 mg dose; for Part B: 2 individuals).

PLG activity levels were monitored during the study. Preliminary data indicated that there were no dose-dependent changes in PLG activity following the single doses of the ds RNAi agent up to 608 mg.

### Example 4: A Randomized, Double-Blind, Placebo-Controlled Study to Investigate the Efficacy and Safety of a ds RNAi Agent for Modulating LPA Expression in Adults with Elevated Lp(a)

Methods: Multicenter studies are designed to evaluate the efficacy and safety of repeat doses of a ds RNAi agent for modulating LPA expression (SEQ ID NOS:3 and 4 for the sense and antisense strands, respectively) in a larger population of individuals with Lp(a) levels that are higher than in Example 3 (i.e., ≥175 nmol/L vs. ≥75 nmol/L). The study is randomized, investigator- and participant-blind, and placebo-controlled.

Doses of the ds RNAi agent are assessed over 540 days in about 254 individuals, who are randomized in a 1:2:2:2:2 ratio to 1 of the arms in Table 6.

TABLE 6
ds RNAi Agent Study Design.

| | | No. of | |
|---|---|---|---|
| Arm | Dose | individuals | Dosing |
| A | 16 mg | 26 | Day 0 at randomization |
| | | | and Day 180 |
| B | 96 mg | 57 | Day 0 at randomization |
| | | | and Day 180 |
| C | 400 mg | 57 | Day 0 at randomization |
| | | | and Day 180 |
| D | 400 mg then 0 mg | 57 | Day 0 at randomization |
| | | | and Day 180 |
| E | 0 mg | 57 | Day 0 at randomization |
| | | | and Day 180 |

All injections are SC administered into the tissue of the abdominal wall. The ds RNAi agent is provided as a 160 mg/mL solution. As in Example 3, placebo is 0.9% NaCl solution for injection (administered in a range from about 0.1 mL to about 2.5 mL).

PD, PK and safety analyses generally are performed as described in Example 3.

Results: An interim analysis shows that the ds RNAi agent lowered Lp(a) similar to that observed in Example 3, with sufficiently sustained Lp(a) lowering to support infrequent dosing. Likewise, the interim analysis shows that the ds RNAi agent was safe and well tolerated.

### Example 5: A Randomized, Double-Blind, Placebo-Controlled Study to Investigate the Effect of a Ds RNAi Agent on the Reduction of Major Adverse Cardiovascular Events in Adults with Elevated Lipoprotein(a) Who have Established Atherosclerotic Cardiovascular Disease or Who are at Risk for a First Cardiovascular Event

Methods: Multicenter studies are designed to investigate the reduction of MACE-4 with repeat doses of a ds RNAi agent for modulating LPA expression (SEQ ID NOS:3 and 4 for the sense and antisense strands, respectively) compared to placebo in adult participants with established ASCVD or adult participants at risk for a first CV event, where MACE-4 is defined as CV death, nonfatal MI, nonfatal stroke and urgent coronary revascularization. The study is randomized, investigator- and participant-blind, and placebo-controlled.

Additional objectives include investigating reduction of Lp(a) levels, reduction of MACE-3 (i.e., CV death, nonfatal MI or nonfatal stroke), reduction of coronary MACE-3 (i.e., coronary heart death, nonfatal MI or urgent coronary revascularization), reduction of MACE-3+MALE (i.e., CV death, nonfatal MI, nonfatal stroke, acute or critical limb ischemia, peripheral artery revascularization or major amputation due to ischemia), reduction of MACE-4 in a subpopulation with established ASCVD and history of a CV event or revascularization, reduction of MACE-4 in a subpopulation at risk for first CV event, reduction of MACE-4 in participants with Lp(a) level≥200 nmol/L, reduction of MI, reduction of CV death and/or reduction of overall mortality.

400 mg doses of the ds RNAi agent are assessed over about 4 years in about 12,500 individuals, who are randomized in a 1:1 ratio to the ds RNAi agent or placebo. The first 3 doses are 6 months apart (Q6M; e.g., Day 0, Day 180 (Week 26) and Day 360 (Week 52)), and all remaining doses are 12 months apart (Q12M); e.g., Week 104 and Week 156) until the end of the study.

All injections are SC administered into the tissue of the abdominal wall. The ds RNAi agent is provided as a 200 mg/mL solution. As in Example 3, placebo is 0.9% NaCl solution for injection (administered in a range from about 0.1 mL to about 2.5 mL).

PD, PK and safety analyses generally are performed as described in Example 3.

### SEQUENCE LISTING

The following nucleotide and/or amino acid sequences are referred to in the disclosure above and are provided below for reference:

-Unmodified Sense Strand (36 nt)
SEQ ID NO: 1
UUGCCAAGCUUGGUCAUCUAGCAGCCGAAAGGCUGC
-Unmodified Antisense Strand (22 nt)
SEQ ID NO: 2
UAGAUGACCAAGCUUGGCAAGG
-Modified Sense Strand (36 nt)
SEQ ID NO: 3
[mUs][mU][mG][mC][mC][mA][mA][fG][fC][fU][fU][mG][mG][mU][mC][mA][mU][mC]
[mU][mA][mG][mC][mA][mG][mC][mC][mG][ademA-GalNAc][ademA-GalNAc][ademA-
GalNAc][mG][mG][mC][mU][mG][mC]
-Modified Antisense Strand (22 nt)
SEQ ID NO: 4
[MePhosphonate-4O-
mUs][fAs][fGs][fA][fU][mG][fA][mC][mC][fA][mA][mG][mC][fU][mU][mG][mG][mC]
[mA][mAs][mGs][mG]

The invention as claimed is:

1. A pharmaceutical composition comprising:
a double-stranded (ds) RNAi agent or its pharmaceutically acceptable salt thereof at a concentration from about 100 mg/mL to about 300 mg/mL in water (H₂O), wherein the pharmaceutical composition is at a pH from about 6.0 to about 8.0, and wherein the ds RNAi agent comprises:
(1) a sense strand comprising a nucleotide sequence of SEQ ID NO:1, and
(2) an antisense strand comprising a nucleotide sequence of SEQ ID NO:2.

2. The pharmaceutical composition of claim 1, wherein the concentration is from about 150 mg/mL to about 170 mg/mL or from about 190 mg/mL to about 210 mg/mL.

3. The pharmaceutical composition of claim 2, wherein the concentration is about 160 mg/mL or about 200 mg/mL.

4. The pharmaceutical composition of claim 1, wherein the sense strand is SEQ ID NO:3.

5. The pharmaceutical composition of claim 1, wherein in the antisense strand is SEQ ID NO:4.

6. The pharmaceutical composition of claim 1, wherein the pH is about 7.0.

7. The pharmaceutical composition of claim 1, wherein the composition is preservative free.

8-11. (canceled)

12. A method of attenuating, preventing and/or treating a disease, disorder and/or condition associated with apolipoprotein(a) gene (LPA) expression in an individual, the method comprising the steps of:
(a) administering to the individual a first dose of a double-stranded (ds) RNAi agent that modulates LPA expression, wherein the first dose is from about 4 mg to about 608 mg, and wherein the ds RNAi agent comprises:
(i) a sense strand comprising SEQ ID NO:1, and
(ii) an antisense strand comprising SEQ ID NO:2; and
(b) administering to the individual one or more subsequent doses of the ds RNAi agent at an interval selected from the group consisting of about every month (Q1M), about every three months (Q3M), about every six months (Q6M), about every nine months (Q9M) and about every twelve months (Q12M) from the first dose or from a previous dose, wherein the one or more subsequent doses are from about 4 mg to about 608 mg.

13. The method of claim 12, wherein the administering is intravenously (IV) or subcutaneously (SC).

14. The method of claim 13, wherein the administering is SC.

15. The method of claim 12, wherein the sense strand is SEQ ID NO:3.

16. The method of claim 12, wherein the antisense strand is SEQ ID NO:4.

17. The method of claim 12, wherein the one or more subsequent doses are the same as the first dose.

18. The method of claim 12, wherein the one or more of the subsequent doses are not the same as the first dose.

19. The method of claim 12, wherein the first dose is selected from the group consisting of about 4 mg, about 12 mg, about 16 mg, about 32 mg, about 96 mg, about 304 mg, about 400 mg and about 608 mg.

20. The method of claim 12, wherein the one or more subsequent doses are independently selected from the group consisting of about 4 mg, about 12 mg, about 16 mg, about 32 mg, about 96 mg, about 304 mg, about 400 mg and about 608 mg.

21-25. (canceled)

26. The method of claim 12, wherein the first dose is about 304 mg, and wherein the one or more subsequent doses are about 304 mg administered about every Q1M, about 304 mg administered about every Q3M, about 304 mg administered about every Q6M, about 304 mg administered about every Q9M or about 304 mg administered about every Q12M from the first dose or from the previous dose.

27. The method of claim 12, wherein the first dose is about 400 mg, and wherein the one or more subsequent doses are about 400 mg administered about every Q1M, about 400 mg administered about every Q3M, about 400 mg administered about every Q6M, about 400 mg administered about every Q9M, or about 400 mg administered about every Q12M from the first dose or from the previous dose.

28. The method of claim 12, wherein the first dose is about 608 mg, and wherein the one or more subsequent doses are about 608 mg administered Q1M, about 608 mg administered Q3M, about 608 mg administered Q6M, about 608 mg administered Q9M, or about 608 mg administered Q12M from the first dose or from the previous dose.

29. The method of claim 12, wherein the first dose is from about 4 mg to about 608 mg, and wherein the one or more subsequent doses are from about 4 mg to about 608 mg and are administered about every Q6M from the first dose or from the previous dose.

30. The method of claim 12, wherein the first dose is from about 4 mg to about 608 mg, wherein a second dose is from about 4 mg to about 608 mg and is administered about Q6M from the first dose, and wherein the one or more subsequent doses are from about 4 mg to about 608 mg and is administered about every Q12M from the second dose or from the previous dose.

31. The method of claim 12, wherein the first dose is from about 4 mg to about 608 mg, and wherein the one or more subsequent doses are from about 4 mg to about 608 mg and are administered about every Q12M from the first dose or from the previous dose.

32. The method of claim 12, wherein the disease, disorder and/or condition associated with LPA expression is selected from the group consisting of atherosclerosis, calcific aortic valve stenosis (CAVS), cardiometabolic diseases, cardiovascular (CV) events including coronary revascularization, CV mortality, cerebrovascular disease, chronic kidney disease, coronary heart disease, dyslipidemia, heart failure, MI and stroke, and peripheral vascular disease.

33. A method of reducing apolipoprotein(a) gene (LPA) expression in an individual having a high lipoprotein(a) (Lp(a)) level, the method comprising the step of:
(a) administering to the individual a first dose of a double-stranded (ds) RNAi agent that modulates LPA expression, wherein the dose is from about 4 mg to about 608 mg, and wherein the ds RNAi agent comprises:
(i) a sense strand comprising SEQ ID NO:1, and
(ii) an antisense strand comprising SEQ ID NO:2,
whereby the Lp(a) level of the individual is reduced by about 90% within about 14 days from the first dose.

34. The method of claim 33, wherein the sense strand is SEQ ID NO:3.

35. The method of claim 33, wherein the antisense strand is SEQ ID NO:4.

36. The method of claim 33, further comprising the step of:
(b) administering to the individual one or more subsequent doses of the ds RNAi agent, wherein one or more subsequent doses are from about 4 mg to about 608 mg, and wherein the one or more subsequent doses are administered from about every 1 month (Q1M) to about every 12 months (Q12M) from the first dose or from a previous dose,
whereby the Lp(a) level of the individual is maintained at the level following the first dose or is reduced further beyond the level following the first dose.

37. The method claim 33, wherein the administering is intravenously (IV) or subcutaneously (SC).

38. The method of claim 37, wherein the administering is SC.

39. The method of claim 36, wherein the one or more subsequent doses are the same as the first dose.

40. The method of claim 36, wherein the one or more subsequent doses are not the same as the first dose.

41. The method of claim 33, wherein the first dose is selected from the group consisting of about 4 mg, about 12 mg, about 16 mg, about 32 mg, about 96 mg, about 304 mg, about 400 mg and about 608 mg.

42. The method of claim 36, wherein the one or more subsequent doses are independently selected from the group consisting of about 4 mg, about 12 mg, about 16 mg, about 32 mg, about 96 mg, about 304 mg, about 400 mg and about 608 mg.

43-47. (canceled)

48. The method of claim 36, wherein the first dose is about 304 mg, and wherein the one or more subsequent doses are about 304 mg administered about every Q1M, about 304 mg administered about every Q3M, about 304 mg administered about every Q6M, about 304 mg administered about every Q9M, or about 304 mg administered about every Q12M from the first dose or from the previous dose.

49. The method of claim 36, wherein the first dose is about 400 mg, and wherein the one or more subsequent doses are about 400 mg administered about every Q1M, about 400 mg administered about every Q3M, about 400 mg administered about every Q6M, about 400 mg administered about every Q9M, or about 400 mg administered about every Q12M from the first dose or from the previous dose.

50. The method of claim 36, wherein the first dose is about 608 mg, and wherein the one or more subsequent doses are about 608 mg administered about every Q1M, about 608 mg administered about every Q3M, about 608 mg administered about every Q6M, about 608 mg administered about every Q9M, or about 608 mg administered about every Q12M from the first dose or from the previous dose.

51. The method of claim 36, wherein the first dose is about 400 mg, and wherein the one or more subsequent doses are about 400 mg and are administered about every Q6M from the first dose or from the previous dose.

52. The method of claim 36, wherein the first dose is about 400 mg, wherein a second dose is about 400 mg and is administered about Q6M from the first dose, and wherein any subsequent dose is about 400 mg and is administered about every Q12M from the second dose or from the previous dose.

53. The method of claim 36, wherein the first dose is about 400 mg, wherein the one or more subsequent doses are 400 mg and are administered about every Q12M from the first dose or from the previous dose.

54-55. (canceled)

56. The method of claim 33, wherein the individual has a disease, disorder and/or condition associated with apolipoprotein(a) gene (LPA) expression selected from the group consisting of atherosclerosis, calcific aortic valve stenosis (CAVS), cardiometabolic diseases, cardiovascular (CV) events including coronary revascularization, CV mortality, cerebrovascular disease, chronic kidney disease, coronary heart disease, dyslipidemia, heart failure, MI and stroke, and peripheral vascular disease.

57-70. (canceled)

71. A method of reducing the risk of a major adverse cardiovascular event (MACE) in an individual, the method comprising:
(a) administering to the individual a first dose of an oligonucleotide that modulates LPA expression, wherein the first dose is from about 4 mg to about 608 mg; and
(b) administering to the individual one or more subsequent doses of the oligonucleotide at an interval selected from the group consisting of about every month (Q1M), about every three months (Q3M), about every six months (Q6M), about every nine months (Q9M) and about every twelve months (Q12M) from the first dose or from a previous dose, wherein the one or more subsequent doses are from about 4 mg to about 608 mg, wherein the individual is at risk for a first cardiovascular event.

72. The method of claim 71, wherein the individual at risk for a first cardiovascular event has a lipoprotein(a) (Lp(a)) level of at least about 75 mmol/L.

73. The method of claim 71, wherein the individual at risk for a first cardiovascular event has a lipoprotein(a) (Lp(a)) level of at least about 100 mmol/L.

74. The method of claim 71, wherein the individual at risk for a first cardiovascular event has a lipoprotein(a) (Lp(a)) level of at least about 125 mmol/L.

75. The method of claim 71, wherein the individual at risk for a first cardiovascular event has a lipoprotein(a) (Lp(a)) level of at least about 150 mmol/L.

76. The method of claim 71, wherein the one or more subsequent doses of the oligonucleotide is at an interval of about every month (Q1M).

77. The method of claim 71, wherein the one or more subsequent doses of the oligonucleotide is at an interval of about every three months (Q3M).

78. The method of claim 71, wherein the one or more subsequent doses of the oligonucleotide is at an interval of about every six months (Q6M).

79. The method of claim 71, wherein the one or more subsequent doses of the oligonucleotide is at an interval of about every twelve months (Q12M).

80. The method of claim 71, wherein the oligonucleotide is a ds RNAi agent.

81. The method of claim 80, wherein the ds RNAi agent comprises:
(i) a sense strand comprising SEQ ID NO:1, and
(ii) an antisense strand comprising SEQ ID NO:2; and

82. The method of claim 80, wherein the ds RNAi agent comprises:
(i) a sense strand comprising SEQ ID NO:3, and
(ii) an antisense strand comprising SEQ ID NO:4; and

83. The method of claim 71, wherein the individual at risk for a first cardiovascular event does not have established atherosclerotic cardiovascular disease.


SEQUENCE LISTING (4 sequences)

SEQ ID NO: 1 - RNA, 36 nt, synthetic construct
  source 1..36: mol_type = other RNA
ttgccaagcttggtcatctagcagccgaaaggctgc

SEQ ID NO: 2 - RNA, 22 nt, synthetic construct
  source 1..22: mol_type = other RNA
tagatgaccaagcttggcaagg

SEQ ID NO: 3 - RNA, 36 nt, synthetic construct; identical to SEQ ID NO: 1
  source 1..36: mol_type = other RNA
  modified_base 1: mod_base = OTHER; note = 2'-O-methyluridine with a 3'-phosphorothioate linkage
  modified_base 2: mod_base = um
  modified_base 3: mod_base = gm
  modified_base 4: mod_base = cm
  modified_base 5: mod_base = cm
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-fluoroguanosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorouridine
  modified_base 12: mod_base = gm
  modified_base 13: mod_base = gm
  modified_base 14: mod_base = um
  modified_base 15: mod_base = cm
  modified_base 16: mod_base = OTHER; note = 2'-O-methyladenosine
  modified_base 17: mod_base = um
  modified_base 18: mod_base = cm
  modified_base 19: mod_base = um
  modified_base 20: mod_base = OTHER; note = 2'-O-methyladenosine
  modified_base 21: mod_base = gm
  modified_base 22: mod_base = cm
  modified_base 23: mod_base = OTHER; note = 2'-O-methyladenosine
  modified_base 24: mod_base = gm
  modified_base 25: mod_base = cm
  modified_base 26: mod_base = cm
  modified_base 27: mod_base = gm
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = gm
  modified_base 32: mod_base = gm
  modified_base 33: mod_base = cm
  modified_base 34: mod_base = um
  modified_base 35: mod_base = gm
  modified_base 36: mod_base = cm

SEQ ID NO: 4 - RNA, 22 nt, synthetic construct
  source 1..22: mol_type = other RNA
  modified_base 1: mod_base = OTHER; note = MePhosphonate-4O-2'-O-methyluridine with a 3'-phosphorothioate linkage
  modified_base 2: mod_base = OTHER; note = 2'-fluoroadenosine with a 3'-phosphorothioate linkage
  modified_base 3: mod_base = OTHER; note = 2'-fluoroguanosine with a 3'-phosphorothioate linkage
  modified_base 4: mod_base = OTHER; note = 2'-fluoroadenosine
  modified_base 5: mod_base = OTHER; note = 2'-fluorouridine
  modified_base 6: mod_base = gm
  modified_base 7: mod_base = OTHER; note = 2'-fluoroadenosine
  modified_base 8: mod_base = cm
  modified_base 9: mod_base = cm
  modified_base 10: mod_base = OTHER; note = 2'-fluoroadenosine
  modified_base 11: mod_base = OTHER; note = 2'-O-methyladenosine
  modified_base 12: mod_base = gm
  modified_base 13: mod_base = cm
  modified_base 14: mod_base = OTHER; note = 2'-fluorouridine
  modified_base 15: mod_base = um
  modified_base 16: mod_base = gm
  modified_base 17: mod_base = gm
  modified_base 18: mod_base = cm
  modified_base 19: mod_base = OTHER; note = 2'-O-methyladenosine
  modified_base 20: mod_base = OTHER; note = 2'-O-methyladenosine with a 3'-phosphorothioate linkage
  modified_base 21: mod_base = OTHER; note = 2'-O-methylguanosine with a 3'-phosphorothioate linkage
  modified_base 22: mod_base = gm
tagatgaccaagcttggcaagg